(19) United States
(12) Patent Application Publication
(10) Pub. No.: US 2012/0165880 A1
(43) Pub. Date: Jun. 28, 2012

(54) BONE FRACTURE TREATMENT DEVICES AND METHODS OF THEIR USE

(76) Inventors: Mark M. Levy, Raanana (IL); Yair Spanier, Pardes-Hanna (IL); Eyal Zylberberg, Kfar Yona (IL); Raphael F. Meloul, Caesarea (IL); John White, Lakeland, TN (US); Dimitri Protopsaltis, Memphis, TN (US)

(21) Appl. No.: 13/321,862

(22) PCT Filed: Jun. 1, 2009
(86) PCT No.: PCT/US09/45858

§ 371 Date: Nov. 22, 2011

Related U.S. Application Data
(60) Provisional application No. 61/057,781, filed on May 30, 2008.

(51) Int. Cl. A61B 17/84

(52) U.S. Cl. 606/300

(57) ABSTRACT

The present disclosure provides for improved devices, systems and methods for stabilizing bones and/or bone segments that have become displaced and/or unstable due to fractures. In exemplary embodiments, the present disclosure provides for improved devices, systems and methods for deploying bone anchoring elements into bone tissue in order to stabilize bones and/or bone segments that have become displaced and/or unstable due to fractures or the like. In one embodiment, a bone treatment assembly includes a shaft configured to extend at least partially into bone tissue. The shaft includes at least one bone anchoring element with a tissue-piercing portion that is selectively displaced or deployed to engage bone tissue. The at least one bone anchoring element may be displaced by one or both of an inner member sized to extend into a lumen in the shaft and/or an outer member that is translatable over the shaft.

### CROSS REFERENCE TO RELATED APPLICATIONS

This application claims the benefit of U.S. Provisional Application No. 61/057,781 filed May 30, 2008, all of which is herein incorporated in its entirety.

### BACKGROUND

1. Technical Field

The present disclosure relates to medical devices and methods and, more specifically, to devices, systems and methods for stabilizing bones and/or bone segments that have become displaced and/or unstable due to fractures.

2. Background Art

Fractures of limb bones have been treated with internal fixation devices, such as plates lying on the surface of a bone, nails running inside the medullary canal of a fractured bone, and/or screws affixing both ends of a fractured bone together. These internal fixation devices may provide reasonable structural rigidity and/or stability to the fractured bone without compromising some of the strain desired to stimulate bone cells.

An intramedullary fixation method is a traditional procedure for treating long bone fractures, which typically involves affixing the bone fracture using intramedullary nails, without disturbing the periosteum of the bone. Such a method may be accomplished in a closed manner, and the fractured bone may be functionally used (including weight bearing) during healing. The surgical approach for insertion of intramedullary nails varies slightly for each bone and is generally well known in the field of orthopedics.

Some of the problems associated with conventional intramedullary fixation methods include lack of rotation stability (e.g., fractured bone segments connected by a nail can rotate relative to each other), lack of longitudinal stability (e.g., fractured bone segments connected by a nail can move relative to each other along an axis of the nail), collapse of the fracture site in some fracture types, and/or undesired backup of nails. In addition, some conventional intramedullary fixation methods may introduce interlocking screws across the nail, creating some disadvantages.

For example, conventional intramedullary fixation nails for long bones may include a rigid structure (hollow or full) that may be locked at their extremes by the addition of screws transversally applied through the bone walls and the nail itself (e.g., interlocking screws). This additional step generally makes the operation longer and more complicated, and may require additional skin incisions and/or longer use of an image intensifier (e.g., X-ray). Furthermore, undesired gaps between the bone ends may originate from the screws, which are permanent unless removed in a new operation. In contaminated fractures, metallic intramedullary nails may propagate contamination through the entire canal, despite attempts at cleaning the fracture site, which may lead to bone infection. While increased stability in an intramedullary fixation device may be desirable, it may also be desired in various situations to remove the fixation device, for example, in the event of discomfort or infection. However, in some situations, the fixation device may be difficult to remove without significantly damaging bone tissue.

Thus, despite efforts to date, a need remains for improved and efficient systems/methods for stabilizing bones and/or bone segments that have become displaced and/or unstable due to fractures. These and other inefficiencies and opportunities for improvement are addressed and/or overcome by the systems and methods of the present disclosure.

### SUMMARY

The present disclosure provides for advantageous devices, systems and methods for stabilizing bones and/or bone segments that have become displaced and/or unstable due to fractures. In exemplary embodiments, the present disclosure provides for advantageous devices, systems and methods for deploying bone anchoring elements into bone tissue (e.g., cancellous or cortical bone) in order to stabilize bones and/or bone segments (e.g., long bone segments) that have become displaced and/or unstable due to fractures or the like.

The present disclosure also provides for a bone treatment assembly including an outer member defining a first lumen; an elongated body member configured and dimensioned to be at least partially disposed within the first lumen, the elongated body member defining a second lumen and at least one opening through the body member, the at least one opening being in communication with the second lumen; at least one bone anchoring element coupled to the body member adjacent the at least one opening, the at least one bone anchoring element being configured and dimensioned to be moveable between at least: (i) a first position at least partially inside the second lumen through the at least one opening to allow positioning of at least a portion of the body member into bone tissue, and (ii) a second position at least partially out of the second lumen and the at least one opening to engage bone tissue adjacent thereto; an inner member configured and dimensioned to be at least partially disposed within the second lumen to engage the at least one anchoring element, thereby moving the at least one anchoring element from the first position to the second position; and wherein after the inner member has moved the at least one anchoring element from the first position to the second position, the outer member is configured and dimensioned to be translatable relative to the body member to thereby move the at least one anchoring element from the second position to the first position.

The present disclosure also provides for a bone treatment assembly wherein the body member and the outer member have circular or non-circular cross-sections. The present disclosure also provides for a bone treatment assembly wherein the outer member is longitudinally or rotationally translatable relative to the body member. The present disclosure also provides for a bone treatment assembly wherein the outer member is selectively separable from the body member.

The present disclosure also provides for a bone treatment assembly wherein the outer member further comprises at least one slot through the outer member, the at least one slot configured and dimensioned to be substantially aligned with the at least one opening through the body member; and wherein the at least one anchoring element is configured and dimensioned to be moved at least partially out of the at least one slot when the at least one slot is substantially aligned with the at least one opening and the at least one anchoring element is moved to the second position. The present disclosure also provides for a bone treatment assembly wherein the outer member includes a surface adjacent to the at least one slot, the surface configured and dimensioned to move the at least one anchoring element from the second position to the first position when the outer member is translated relative to the body member. The present disclosure also provides for a bone treatment assembly wherein the at least one slot includes an extension extending from the at least one slot, and wherein the outer member is configured and dimensioned to be rotationally and longitudinally translated such that the at least one anchoring element is positioned in the extension to maintain the at least one anchoring element in the second position.

The present disclosure also provides for a bone treatment assembly wherein the at least one anchoring element includes a hinged portion extending along a first axis and an engaging portion formed by bending an end of the hinged portion along a second axis, the second axis being substantially perpendicular to the first axis. The present disclosure also provides for a bone treatment assembly wherein the at least one anchoring element includes a hinged portion extending along a first axis and an engaging portion formed by bending an end of the hinged portion along a second axis, the second axis being angled relative to the first axis. The present disclosure also provides for a bone treatment assembly wherein the at least one anchoring element is formed out of the body member. The present disclosure also provides for a bone treatment assembly wherein the at least one anchoring element is at least partially plastically deformable. The present disclosure also provides for a bone treatment assembly wherein the inner member is configured and dimensioned to expand within the second lumen to engage and move the at least one anchoring element from the first position to the second position. The present disclosure also provides for a bone treatment assembly wherein the inner member is configured and dimensioned to slide within the second lumen to engage and move the at least one anchoring element from the first position to the second position. The present disclosure also provides for a bone treatment assembly wherein the inner member is configured and dimensioned to translate within the second lumen relative to the body member in a first direction to engage and move the at least one anchoring element from the first position to the second position. The present disclosure also provides for a bone treatment assembly wherein the inner member is configured and dimensioned to translate within the second lumen relative to the body member in a second direction to move the at least one anchoring element from the second position to the first position.

The present disclosure also provides for a bone treatment assembly wherein at least one anchoring element further includes a sliding member, the sliding member configured and dimensioned to be received in a guiding slot in a wall of the inner member. The present disclosure also provides for a bone treatment assembly wherein the body member is at least partly comprised of a mesh. The present disclosure also provides for a bone treatment assembly wherein the body member or the outer member is at least partially coated with a pharmaceutical agent. The present disclosure also provides for a bone treatment assembly wherein the at least one anchoring element has a first end having a tissue-piercing portion and a second end that is coupled to a wall of the body member. The present disclosure also provides for a bone treatment assembly wherein the engaged inner member maintains the at least one anchoring element in the second position and prevents the at least one anchoring element from moving to the first position; and wherein the inner member is moved out of engagement with the at least one anchoring element prior to translating the outer member relative to the body member to move the at least one anchoring element from the second position to the first position.

The present disclosure also provides for a bone treatment assembly wherein the inner member is removable from the second lumen after the at least one anchoring element has moved from the first position to the second position. The present disclosure also provides for a bone treatment assembly wherein the outer member includes a plurality of outer member sections coupled together; wherein the body member includes a plurality of body member sections coupled together; and wherein the inner member includes a plurality of inner member sections coupled together. The present disclosure also provides for a bone treatment assembly wherein the outer member or the body member may be incrementally lengthened in the bone tissue. The present disclosure also provides for a bone treatment assembly further including an inserter member releasably coupled to the inner member or the outer member, the inserter member having a drive mechanism; and wherein the drive mechanism of the inserter member is configured and dimensioned to allow a user to move the inner member into or out of engagement with the at least one anchoring element. The present disclosure also provides for a bone treatment assembly wherein the drive mechanism of the inserter member is configured and dimensioned to allow a user to translate the outer member relative to the body member. The present disclosure also provides for a bone treatment assembly wherein the inserter member further includes a ratchet, the ratchet being configured and dimensioned to allow a user to actuate the drive mechanism. The present disclosure also provides for a bone treatment assembly wherein the inserter member further includes an attachable sleeve, the attachable sleeve configured and dimensioned to assist a user to place anchors or screws through an end of the body member.

The present disclosure also provides for a bone treatment assembly further including an extension member releasably coupled to the outer member or to the body member, the extension member being configured and dimensioned to extend at least partially through a bone or through an entry portal hole of the bone after the body member has been positioned substantially inside the bone tissue; and wherein the outer diameter of the extension member is smaller than the outer diameter of the outer member. The present disclosure also provides for a bone treatment assembly wherein after the inner member has moved the at least one anchoring element from the first position to the second position, the body member is configured and dimensioned to be translated relative to the outer member to thereby move the at least one anchoring element from the second position to the first position.

The present disclosure also provides for a method for treating a bone including inserting a device at least partially into bone tissue, the device having: (i) an outer member defining a first lumen, (ii) an elongated body member at least partially disposed within the first lumen, the elongated body member defining a second lumen and at least one opening through the body member, the at least one opening being in communication with the second lumen, and (iii) at least one bone anchoring element coupled to the body member adjacent the at least one opening, the at least one bone anchoring element being configured and dimensioned to be moveable between at least (a) a first position at least partially inside the second lumen through the at least one opening to allow positioning of at least a portion of the body member into bone tissue, and (b) a second position at least partially out of the second lumen and the at least one opening to engage bone tissue adjacent thereto; inserting an inner member at least partially into the second lumen of the body member to move the at least one bone anchoring element from the first position to the second position to engage bone tissue adjacent thereto. The present disclosure also provides for a method for treating a bone further including the step of translating the outer member relative to the elongated body member to disengage the at least one bone anchoring element from the bone tissue and to thereby move the at least one bone anchoring element from the second position to the first position. The present disclosure also provides for a method for treating a bone further including the step of translating the elongated body member relative to the outer member to disengage the at least one bone anchoring element from the bone tissue and to thereby move the at least one bone anchoring element from the second position to the first position.

The present disclosure also provides for a bone treatment assembly including an outer member defining a lumen and at least one opening through the outer member, the at least one opening being in communication with the lumen; an elongated body member configured and dimensioned to be at least partially disposed within the lumen; at least one bone anchoring element rotatably coupled to a connector that is coupled to the body member, the at least one anchoring element being configured and dimensioned to be moveable between at least: (i) a first position inside the lumen, and (ii) a second position at least partially out of the at least one opening; wherein translation of the outer member relative to the body member to a position where the at least one opening is substantially aligned with the at least one anchoring element causes the at least one anchoring element to rotate relative to the connector to the second position at least partially out of the at least one opening. The present disclosure also provides for a bone treatment assembly wherein the at least one anchoring element or the connector is spring-tensioned to facilitate controlled movement of the at least one anchoring element. The present disclosure also provides for a bone treatment assembly wherein after the at least one anchoring element has moved to the second position, translation of the outer member over the at least one anchoring element in the second position causes the at least one anchoring element to rotate around the connector to the first position inside the lumen. The present disclosure also provides for a bone treatment assembly wherein the at least one opening has at least one tapered edge to facilitate the rotation of the at least one anchoring element. The present disclosure also provides for a bone treatment assembly wherein the outer member is longitudinally or rotationally translatable relative to the body member. The present disclosure also provides for a bone treatment assembly wherein the at least one anchoring element further includes two joined rotating member, each rotating member being rotatably coupled to a connector coupled to the body member.

The present disclosure also provides for a bone treatment assembly including an elongated body member defining a first lumen and at least one opening through the body member, the at least one opening being in communication with the first lumen; an inner member configured and dimensioned to be at least partially disposed within the first lumen, the inner member defining: (i) a second lumen, (ii) at least one guiding slot in a wall of the inner member, and (iii) at least one receiving slot through the wall of the inner member, the at least one receiving slot being in communication with the second lumen; at least one bone anchoring element coupled to the body member adjacent the at least one opening, the at least one anchoring element including a sliding member at least partially disposed within the first lumen and positioned at least partially in the guiding slot, the at least one anchoring element being configured and dimensioned to be moveable between at least: (i) a first position at least partially inside the first lumen and the receiving slot, and (ii) a second position at least partially out of the first lumen and the receiving slot to engage bone tissue adjacent thereto; wherein translation of the inner member relative to the body member in a first direction causes the guiding slot to engage and move the sliding member, thereby moving the at least one anchoring element from the first position to the second position. The present disclosure also provides for a bone treatment assembly wherein after the at least one anchoring element has moved to the second position, translation of the inner member relative to the body member in a second direction causes the guiding slot to engage and move the sliding member, thereby moving the at least one anchoring element from the second position to the first position.

The present disclosure also provides for a bone treatment assembly including an outer member defining a first lumen and at least one first opening through the outer member, the at least one first opening being in communication with the first lumen; an elongated body member configured and dimensioned to be at least partially disposed within the first lumen, the elongated body member defining a second lumen and at least one second opening through the elongated body member, the at least one second opening being in communication with the second lumen; an inner member configured and dimensioned to be at least partially disposed within the second lumen, the inner member having at least one inner recess; at least one bone anchoring element coupled to the elongated body member adjacent the at least one second opening, the at least one anchoring element being configured and dimensioned to be moveable between at least: (i) a first position at least partially within the at least one inner recess, and (ii) a second position at least partially out of the first opening; wherein translation of the inner member relative to the body member in a first direction causes the inner member to engage and move the at least one anchoring element from the first position to the second position.

The present disclosure also provides for a bone treatment assembly wherein after the at least one anchoring element has moved to the second position, translation of the inner member relative to the body member in a second direction causes the inner member to move the at least one anchoring element from the second position to the first position. The present disclosure also provides for a bone treatment assembly wherein after the at least one anchoring element has moved to the second position, the inner member is configured and dimensioned to allow a user to translate the inner member relative to the body member in a second direction to substantially align the at least one inner recess with the first opening; and wherein translation of the outer member relative to the body member and the inner member causes the outer member to move the at least one anchoring from the second position to the first position. The present disclosure also provides for a bone treatment assembly wherein after the at least one anchoring element has moved to the second position, the inner member is configured and dimensioned to allow a user to translate the inner member relative to the body member in a second direction to substantially align the at least one inner recess with the first opening; and wherein translation of the body member relative to the outer member and the inner member causes the outer member to move the at least one anchoring from the second position to the first position.

The present disclosure also provides for a bone treatment assembly wherein when the at least one anchoring element is in the first position, the at least one anchoring element is not protruding through the first opening. The present disclosure also provides for a bone treatment assembly wherein the inner member moves the at least one anchoring element from the first position to the second position via a cam mechanism. The present disclosure also provides for a bone treatment assembly wherein the engaged inner member maintains the at least one anchoring element in the second position and prevents the at least one anchoring element from moving to the first position. The present disclosure also provides for a bone treatment assembly further including a displacement member, the displacement member configured and dimensioned to allow a user to cause the inner member to translate relative to the body member to move the at least one anchoring element to the first or second position. The present disclosure also provides for a bone treatment assembly wherein the displacement member is releasably attached to an end of the inner member or to an end of the outer member. The present disclosure also provides for a bone treatment assembly wherein at least a portion of the displacement member is disposed within the first lumen or second lumen. The present disclosure also provides for a bone treatment assembly further including a screw guide positioned between the displacement member and the inner member, the screw guide configured and dimensioned to contact or engage the inner member. The present disclosure also provides for a bone treatment assembly wherein at least a portion of the screw guide is disposed within the first or second lumen.

The present disclosure also provides for a bone treatment assembly further including a screw guide at least partially disposed in the first or second lumen; wherein the screw guide allows a user to insert at least one screw or anchor through the screw guide and into engagement with bone tissue; and wherein the screw guide is removable and replaceable. The present disclosure also provides for a bone treatment assembly wherein the outer member, inner member and body member are configured and dimensioned to allow a user to dispose a guide wire at least partially within the first or second lumen; and wherein the at least one anchoring element is configured and dimensioned to allow a user to dispose a guide wire at least partially within the first or second lumen. The present disclosure also provides for a bone treatment assembly wherein after the guide wire is disposed within the first or second lumen, the at least one anchoring element may be moved to the first or second position. The present disclosure also provides for a bone treatment assembly wherein the inner member is longitudinally or rotationally translatable relative to the body member. The present disclosure also provides for a bone treatment assembly wherein the at least one anchoring element includes a hinged portion extending along a first axis and an engaging portion formed by bending an end of the hinged portion along a second axis, the second axis being angled relative to the first axis. The present disclosure also provides for a bone treatment assembly wherein the at least one anchoring element is formed out of the body member. The present disclosure also provides for a bone treatment assembly wherein the body member or the outer member is at least partly comprised of a mesh.

The present disclosure also provides for a bone treatment assembly further including an inserter member releasably coupled to the inner member or the outer member, the inserter member having a drive mechanism; and wherein the drive mechanism of the inserter member is configured and dimensioned to allow a user to translate the inner member relative to the body member. The present disclosure also provides for a bone treatment assembly wherein the inserter member further includes a ratchet, the ratchet being configured and dimensioned to allow a user to actuate the drive mechanism. The present disclosure also provides for a bone treatment assembly wherein the inserter member further includes an attachable sleeve, the attachable sleeve configured and dimensioned to assist a user to place anchors or screws through an end of the body member. The present disclosure also provides for a bone treatment assembly further including an extension member releasably coupled to the outer member or to the body member, the extension member being configured and dimensioned to extend at least partially through a bone or through an entry portal hole of the bone after the body member has been positioned substantially inside the bone; and wherein the outer diameter of the extension member is smaller than the outer diameter of the outer member.

Additional advantageous features, functions and applications of the disclosed systems and methods of the present disclosure will be apparent from the description which follows, particularly when read in conjunction with the appended figures.

### BRIEF DESCRIPTION OF THE DRAWINGS

To assist those of ordinary skill in the art in making and using the disclosed systems and methods, reference is made to the appended figures, wherein:

FIG. 1 is a perspective view of a bone treatment assembly constructed in accordance with an exemplary embodiment of the present disclosure, particularly showing a bone treatment shaft or body member with an outer member disposed thereon and an inner member;

FIG. 2 is a sectional perspective view of the bone treatment assembly in FIG. 1, particularly showing an outer member disposed on a bone treatment shaft or body member;

FIG. 3 is a sectional perspective view of the bone treatment assembly in FIG. 1, particularly showing an inner member in a bone treatment shaft or body member;

FIG. 4 is a sectional perspective view of the bone treatment assembly in FIG. 1, particularly showing an outer member translated over a bone treatment shaft or body member;

FIG. 5 is a sectional perspective view of an alternative embodiment of a bone treatment assembly of the present disclosure, particularly showing an alternative embodiment of anchoring elements coupled to the bone treatment shaft or body member;

FIGS. 6A and 6B are sectional perspective views of an alternative embodiment of a bone treatment assembly of the present disclosure, particularly showing an alternative embodiment of slots on an outer member;

FIGS. 7A and 7B are combined sectional perspective and cross-sectional views of an alternative embodiment of a bone treatment assembly of the present disclosure, particularly showing an alternative embodiment of anchoring elements coupled to the bone treatment shaft or body member;

FIG. 8A is a combined sectional perspective and cross-sectional view of an alternative embodiment of a bone treatment assembly of the present disclosure, particularly showing another alternative embodiment of anchoring elements coupled to the bone treatment shaft or body member;

FIG. 8B is a cross-sectional view taken along the line 8B in FIG. 8A;

FIG. 8C illustrates the embodiment shown in FIG. 8B with deployed anchoring elements coupled to the bone treatment shaft or body member;

FIGS. 9A and 9B are combined sectional perspective and cross-sectional views of an alternative embodiment of a bone treatment assembly of the present disclosure, particularly showing another alternative embodiment of anchoring elements coupled to the bone treatment shaft or body member;

FIGS. 10A and 10B are cross-sectional views of an alternative embodiment of a bone treatment assembly of the present disclosure, particularly showing another alternative embodiment of an anchoring element coupled to the bone treatment shaft or body member;

FIG. 11 is a perspective view of an alternative embodiment of a bone treatment assembly according to the present disclosure;

FIGS. 12A and 12B are perspective views of the bone treatment assembly in FIG. 1 inserted in a medullary canal;

FIG. 13 is a perspective view of an alternative embodiment of a bone treatment assembly according to the present disclosure;

FIG. 14 is a disassembled view of the embodiment of the bone treatment assembly in FIG. 13;

FIG. 14A is a cross-sectional view of an outer shaft taken along the line 14A-14A in FIG. 14;

FIGS. 15A and 15B are longitudinal cross-sectional views of a portion of the embodiment of the bone treatment assembly in FIG. 13;

FIG. 16 is a cross-sectional view of an alternative embodiment of the bone treatment assembly in FIG. 13;

FIG. 17, is a partial perspective view of the alternative embodiment of the bone treatment assembly shown in FIG. 16;

FIG. 18 is a partial perspective view of the embodiment of the bone treatment assembly in FIG. 13;

FIGS. 19-23 are frontal views of exemplary embodiments of an inserter member for a bone treatment assembly;

FIG. 24 is a side view of an exemplary embodiment of an inserter member for a bone treatment assembly;

FIGS. 25A-25C are partial cross-sectional views of an embodiment of a bone treatment assembly according to the present disclosure;

FIGS. 26A-26B are partial cross-sectional views of an embodiment of a bone treatment assembly according to the present disclosure; and

FIG. 27 is a side view of an embodiment of a bone treatment assembly according to the present disclosure.

### DETAILED DESCRIPTION

The present disclosure provides for improved devices, systems and methods for stabilizing bones and/or bone segments that have become displaced and/or unstable due to fractures. In exemplary embodiments, the present disclosure provides for improved devices, systems and methods for deploying bone anchoring elements into bone tissue (e.g., cancellous or cortical bone) in order to stabilize bones and/or bone segments (e.g., long bone segments) that have become displaced and/or unstable due to fractures or the like.

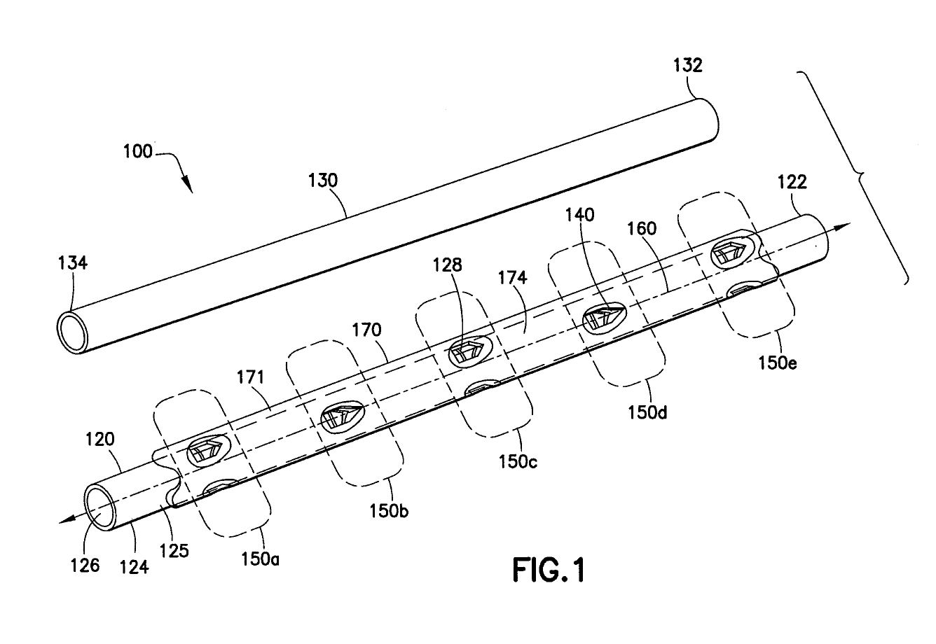

Referring now to FIGS. 1-4, a bone treatment assembly 100, in accordance with one embodiment of the present disclosure will now be described. The assembly 100 generally includes a bone treatment shaft or body member 120, an inner member 130 that may facilitate anchoring of the bone treatment shaft or body member 120 along and/or inside a medullary canal of a bone, and an outer member 170 that is translatable over or relative to the bone treatment shaft or member 120 (FIG. 1). In exemplary embodiments, the assembly includes an elongated bone treatment shaft or member 120 that is configured and dimensioned to extend at least partially into bone or bone tissue, an elongated inner member 130 that is configured and dimensioned to facilitate anchoring of the bone treatment shaft or member 120 along and/or inside a medullary canal of a bone, and an elongated outer member 170 that is configured and dimensioned to: (i) extend at least partially into bone or bone tissue, and (ii) be translatable over or relative to the elongated treatment shaft or body member 120. For example, the treatment shaft 120, inner member 130 and/or outer member 170 may take the form of a cannula, although the present disclosure is not limited thereto. Rather, the treatment shaft or body member 120, inner member 130 and/or outer member 170 may take a variety of forms.

In general, the bone treatment shaft or body member 120 has a first end 122, a second end 124, and a shaft wall 125 defining a lumen 126 that extends along a longitudinal axis 160 between the first and the second ends 122, 124. The inner member 130 has a first end 132 and a second end 134 and is sized such that it can be inserted into or disposed within the lumen 126 of the bone treatment shaft 120 during use. The outer member 170 typically includes a wall 171 defining a lumen 174 (shown in phantom) extending longitudinally through the wall 171 and sized such that the treatment shaft 120 may positioned in or disposed within the lumen 174. In exemplary embodiments, the outer member 170 and/or the treatment shaft or body member 120 is configured to interface with the medullary canal of a bone during use.

The outer surface of the treatment shaft 120, inner member 130 and/or outer member 170 may be finished or the like in a variety of different ways (e.g., polished, bead blasted, fluted, etc.). Additionally, the outer surface of the treatment shaft 120, inner member 130 and/or outer member 170 may include a coating (e.g., a biological coating) such as, for example, an antibiotic coating, a hydroxyl appatite coating, a BMP coating, a steroid coating, etc. In exemplary embodiments, the bone treatment assembly 100 may also incorporate biologicals or the like (e.g., antibiotics, hydroxyl appatite, BMP's, steroids, etc.) into the assembly 100 via pockets, grooves and/or reservoirs or the like.

The bone treatment shaft or body member 120, inner member 130, and/or outer member 170 may be fabricated from a variety of materials, such as, for example, biocompatible materials, plastics, polymers, metals, alloys, ceramics, titanium, stainless steel, carbon fiber, PEEK, resorbable materials, shape memory materials and/or biological materials. For example, the bone treatment shaft or body member 120, inner member 130, and/or outer member 170 may be fabricated from a bioabsorbable material, a tissue engineered material, a shape memory alloy or polymer, such as, without limitation, nitinol, or other resilient materials, such as stainless steel or a titanium alloy, or combinations of both bioabsorbable, or tissue-engineered, and/or non-bioabsorbable materials. The bone treatment shaft 120, inner member 130, and/or outer member 170 may also be fabricated from a homogenous material or from materials of differing nature/properties (e.g., heterogeneous materials). Preferably, the bone treatment shaft 120 and/or the outer member 170 is rigid enough or rigid for the required period of time to provide stability to the fractured and/or unstable bone in which it will be anchored.

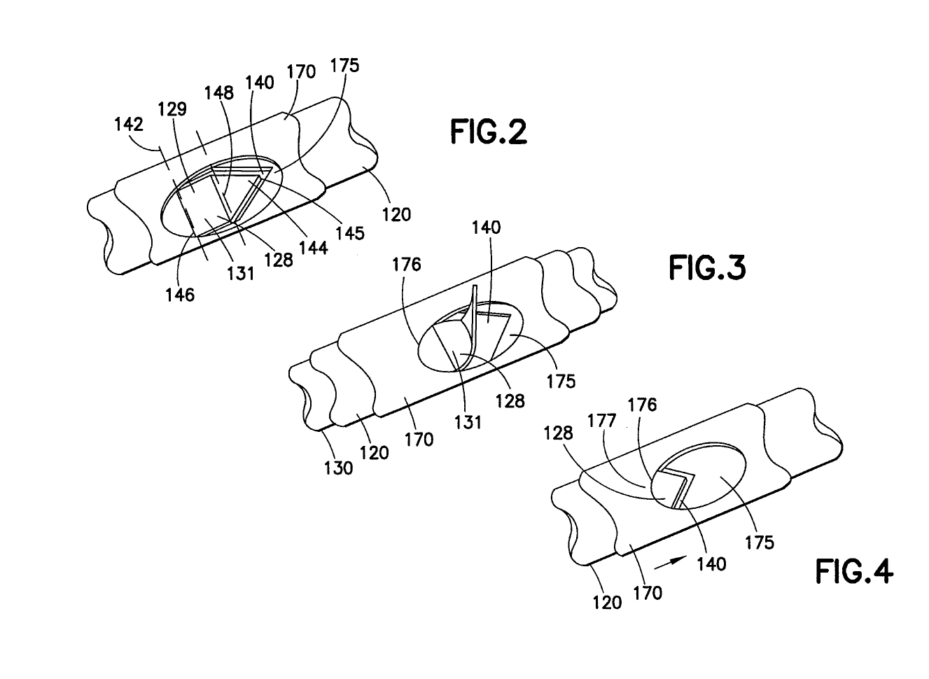

In general, the bone treatment shaft or body member 120 includes at least one bone anchoring element 128. In exemplary embodiments, the bone treatment shaft 120 includes a plurality of bone anchoring elements 128 coupled (e.g., hingedly coupled) and/or connected to the shaft wall 125 and a plurality of respective openings 140 formed through the shaft wall 125. In one embodiment, each anchoring element 128 has a first end 144 having a tissue-piercing portion 145 (e.g., a sharp, tissue-piercing tip 145 or the like), and a second end 146 that is coupled, secured and/or connected to the wall 125 of the bone treatment shaft or body member 120 (FIG. 2). In the illustrated embodiment, the first end 144 of the anchoring element 128 is closer to the first end 122 of the bone treatment shaft 120, and the second end 146 of the anchoring element 128 is closer to the second end 124 of the bone treatment shaft 120, although the present disclosure is not limited thereto. In alternative embodiments, instead of a single sharp tip 145, the anchoring elements 128 may have a plurality of sharp tips. In other embodiments, the anchoring elements 128 may have blunt and/or sharp side sections and/or blunt tips. The anchoring elements 128 may also have a variety of shapes and/or sizes, such as, for example, triangular shapes, spiked shapes, rounded shapes, arrowhead shapes, teeth shapes, curved teeth shapes, roughened surfaces, along with various other shapes/sizes/surfaces. The anchoring elements 128 may have the same or similar shapes and/or lengths, or the anchoring elements 128 may have different shapes and/or lengths.

In exemplary embodiments, the anchoring elements 128 may have various orientations and/or angles of penetration when they are displaced or deployed in the bone tissue (e.g., cancellous or cortical bone). The anchoring elements 128 may be deployed and/or displaced via the same or similar mechanism/method, or the anchoring elements may be deployed and/or displaced via differing mechanisms/methods. The anchoring elements 128 may be deployed/displaced at substantially the same or similar period of time, or the anchoring elements may be deployed/displaced at various separate periods of time (e.g., some anchoring elements 128 may be deployed prior to other anchoring elements 128 being deployed). In exemplary embodiments of the present disclosure, each anchoring element 128 may be un-deployed after being deployed. In one embodiment of the present disclosure, each anchoring element 128 may be un-deployed after being deployed by reversing the process/method used to deploy the anchoring element 128.

In one embodiment, the anchoring elements 128 are formed out of or fabricated from the shaft wall 125. For example, each anchoring element 128 and each respective opening 140 are made by cutting through the wall 125 of the bone treatment shaft 120, such that a cut portion 129 (defined by a profile of the cut) of the wall 125 may be bent. For example, the cutting can be accomplished using a laser beam or a mechanical cutter or the like. The first end 144 of the cut portion 129 is then bent along a first line 148 and away from the longitudinal axis 160 to create an engaging portion (e.g., a spike or thorn) that points radially away from the axis 160. The first line 148 may be substantially perpendicular relative to the longitudinal axis 160, as illustrated in FIG. 2, or alternatively, the first line 148 may be angled relative to the longitudinal axis 160 (e.g., non-parallel to axis 160). A hinged portion 131 may then be formed by bending the cut portion 129 along a second line 142, and into the lumen 126. It should be noted that instead of the cut profile shown, in alternative embodiments, different cut profiles can be used to create different shapes for the openings 140 and the anchoring elements 128. Alternatively, instead of forming each anchoring element 128 out of a portion of the wall 125, the anchoring elements 128 may be separately manufactured/fabricated and then secured, attached and/or coupled to the bone treatment shaft or body member 120 using, for example, various methods/mechanisms, including without limitation, gluing, welding, machining, hinges, etc., or any other suitable adhesives and/or methods.

In exemplary embodiments of the present disclosure and as depicted in FIG. 1, the bone treatment shaft 120 includes five sets 150a-e of the anchoring elements 128 disposed or distributed along the shaft wall 125 of the bone treatment shaft 120, with each of the sets 150a-e having three anchoring elements 128 circumferentially disposed or distributed along the shaft wall 125 of the bone treatment shaft 120. In alternative embodiments, instead of having five sets of three anchoring elements 128, the bone treatment shaft 120 can include any number of sets, and each of the sets can include any number of anchoring elements 128. In one embodiment, the anchoring elements 128 of the sets 150 are circumferentially aligned. Alternatively, the anchoring elements 128 may be staggered, or instead of having a regular or a well-defined pattern, the anchoring elements 128 may be randomly disposed or distributed along the length of the bone treatment shaft 120. In any of the embodiments described herein, the anchoring elements 128 can have the same or different shapes and/or sizes.

In one embodiment, the inner member 130 is an elongated inner member having a cross-sectional dimension smaller than a cross-sectional dimension of the bone treatment shaft lumen 126, thereby allowing the inner member 130 to be inserted into or disposed within the lumen 126. In general, the cross-sectional dimension of the inner member 130 should also be large enough such that when the inner member 130 is positioned or inserted within the lumen 126, an exterior surface of the inner member 130 engages the anchoring elements 128 and causes at least one of the anchoring elements 128 to be displaced or deployed through the respective openings 140 of the bone treatment shaft 120, as shown in FIG. 3. In exemplary embodiments, the engaged inner member 130 maintains the deployed anchoring element(s) in the deployed position, and prevents the deployed anchoring element(s) from moving back to the pre-deployed position. In general, the anchoring elements 128 are also further deployed or displaced through respective slots or openings or the like on the outer member 170 and into engagement with bone or bone tissue, as described below in further detail.

In one embodiment, the cross-sectional dimension of the inner member 130 is slightly smaller (e.g., smaller by about ⅛″) than the cross-sectional dimension of the lumen 126, thereby allowing the inner member 130 to engage and deploy/displace the anchoring elements 128 out of the lumen 126 through the respective openings 140. In another embodiment, the cross-sectional dimension of the inner member 130 can be made smaller to control the degrees of deployment of the anchoring elements 128.

The anchoring elements 128 may be configured to undergo plastic deformation as they are deployed, or alternatively, the anchoring elements 128 may be configured to undergo elastic deformation as they are deployed. Also, in other embodiments, the anchoring elements 128 may undergo deformation that is in part elastic and in part plastic. In other embodiments, shape memory materials can be used, thereby allowing the anchoring elements 128 to be deployed without the use of an inner member 130. In still other embodiments, the inner member 130 can be made out of, for example, bone graft tissue, natural or synthetic bioabsorbable material, or tissue engineering material, as a scaffold for cell seeding or for the conduction or the induction of natural bone tissue. In some cases, a rigid inner member 130 can be replaced by an element made out of one of the previously mentioned materials for more biological integration while supporting and/or deploying the anchoring elements 128. Also, both the shaft wall 125 and the inner member 130 can be made of the same type of material for full integration, for example.

In exemplary embodiments, the outer member 170 is translatable relative to the bone treatment shaft 120 when the bone treatment shaft 120 is positioned in or disposed within the outer member lumen 174. In particular, the outer member 170 may be translatable over the deployed anchoring elements 128 to displace or depress the anchoring elements 128 in the bone treatment shaft lumen 126, as shown in FIG. 4 (e.g., after the inner member 130 has deployed the anchoring elements 128 through the openings 140). Thus, in exemplary embodiments, when the anchoring elements 128 are positioned/deployed in bone tissue, the outer member 170 may be translatable over the bone treatment shaft 120, including the anchoring elements 128, to dislodge the anchoring elements 128 from the bone tissue and depress the anchoring elements 128 in the bone treatment shaft lumen 126. In this manner, the bone treatment shaft 120 is retrievable from the bone tissue site, along with the outer member 170, without the anchoring elements 128 disrupting such retrieval. The outer member 170 may be sufficiently rigid to effectively dislodge the anchoring elements 128 from bone tissue. The inner member 130 may or may not be removed from the bone treatment shaft lumen 126 before the outer member 170 is translated over or relative to the bone treatment shaft 120 to dislodge the anchoring elements 128 from the bone tissue and depress the anchoring elements 128 in the bone treatment shaft lumen 126.

In the illustrated embodiments in FIGS. 1-4, the outer member 170 includes a plurality of slots or openings 175, wherein the position of the slots 175 substantially corresponds to the positions of the openings 140 on the bone treatment shaft 120. The slots 175 may be substantially aligned with the openings 140 on the bone treatment shaft 120 to form an open position, such that the anchoring elements 128 can protrude through both the slots 175 and the openings 140. In this embodiment, the outer member 170 is translatable relative to the bone treatment shaft 120 in a direction such that an edge 176 of each slot 175 engages a respective anchoring element 128. As translation of the outer member 170 is continued in the same direction, a continuous surface 177 adjacent to the engaging edge 176 of each slot 140 translates over each anchoring element 128 to depress the anchoring element 128 into the bone treatment shaft lumen 126, forming a closed position (FIG. 4). FIG. 4 illustrates the outer member 170 longitudinally translated from the second end 124 of the bone treatment shaft or body member 120 toward the first end 122, such that the continuous surface 177 first translates over the hinged portion 131 of the anchoring element 128. The slots 175 and the openings 140 may take the form of a variety of geometries (e.g., round, oval, square, etc.), and they may be positioned at variable locations along the longitudinal axis of the assembly 100. The anchoring elements 128 may or may not intimately fit in the slots 175 and/or openings 140 once deployed.

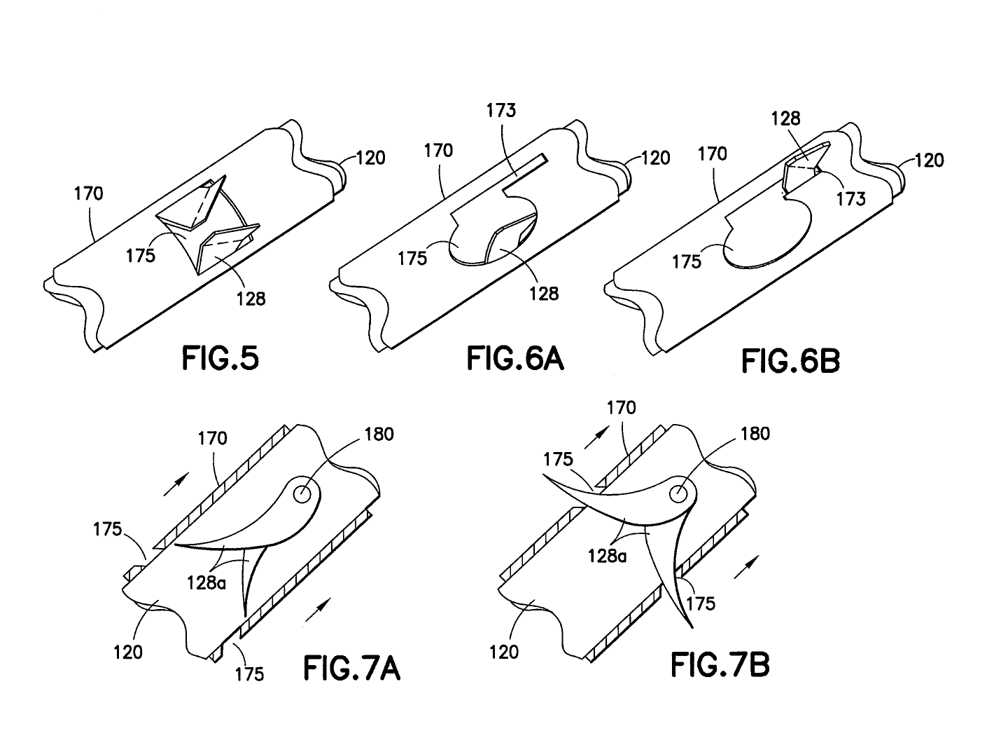

While FIGS. 1-4 illustrate the anchoring elements 128 aligned in the same direction, the anchoring elements 128 may also be aligned in multiple directions. For example, the anchoring elements 128 may be bent at different angles relative to the longitudinal axis 160, as shown in FIG. 5. The anchoring elements 128 may still be structured, however, to allow the outer member 170 to translate over the anchoring elements 128.

As an alternative to longitudinal translation, the outer member 170 may be rotationally translated to form the closed position. For example, this embodiment may be particularly used when the first line 148 along which the cut portion 129 of the anchoring elements 128 are bent is angled relative to the longitudinal axis 160. In this manner, the engaging edge 176 of each slot 175 engages a side edge of each anchoring element 128 to initiate the displacement or un-deployment of the anchoring element 128.

The slots 175 may further include additional sections for positioning the anchoring elements 128 therein to help maintain the anchoring elements 128 in a deployed position, which may require both longitudinal and rotational translation. As an example, FIGS. 6A and 6B illustrate an embodiment in which the slots 175 have a narrow extension 173 that extends from each slot 175. The outer member 170 is longitudinally translated to align the slots 175 with the openings 140 to form the open position, so the anchoring elements 128 may be deployed through the slots 175, as shown in FIG. 6A. The outer member 170 may then be both rotationally and longitudinally translated such that the anchoring element 128 is positioned in the extension 173, as shown in FIG. 6B.

FIGS. 7A and 7B illustrate another embodiment in which the anchoring elements 128 are deployed through the slots 175 by rotating around a connector 180, such as, for example, a bolt, that couples the anchoring elements 128 to the bone treatment shaft 120. In this embodiment, each of the anchoring elements 128 includes one or more rotating members 128a, such as a pair of rotating members 128a, as illustrated in FIGS. 7A and 7B. The rotating members 128a may be straight or curved. Also, each rotating member 128a, in cooperation with the connector 180, may be spring-tensioned to facilitate controlled movement of the rotating elements 128a. Each connector 180 may couple one rotating member 128a or a multiple of rotating members 128a, as shown in FIGS. 7A and 7B, to the bone treatment shaft 120.

As shown in FIG. 7A, the anchoring elements 128 may be positioned on the bone treatment shaft 120 and in the lumen 174 of the outer member 170, until the outer member 170 is translated to substantially align the slots 175 with the anchoring elements 128. The rotating members 128a then rotate outwardly around the connectors 180 and out of the slots 175, as shown in FIG. 7B. When the outer member 170 is translated over the deployed anchoring elements 128, the anchoring elements 128 rotate inwardly back through the slots 175 to be received in the outer member lumen 174. The slots 175 may have tapered edges to facilitate the rotation of the rotating members 128a. Notably, the illustrated embodiment demonstrates longitudinal translation of the outer member 170 to allow deployment and depression of the anchoring elements 128, but the anchoring elements 128 and the outer member 170 may also be configured to facilitate rotational translation of the outer member 170. Further, as an inner member 130 may not be used to deploy the anchoring elements 128 in this embodiment, the bone treatment shaft 120 may or may not include a lumen 126 for receiving an inner member 130.

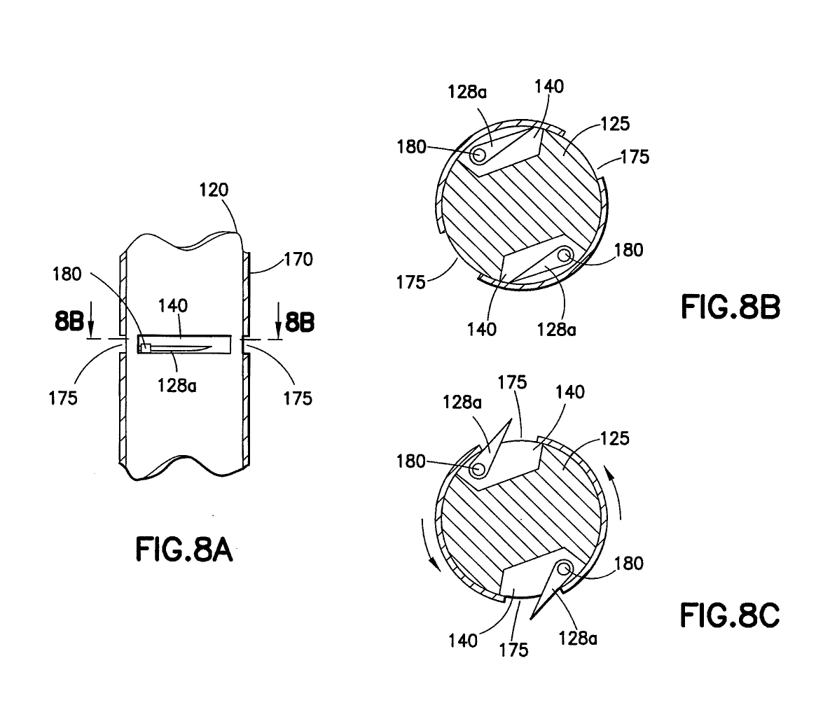

In an alternative embodiment, illustrated in FIGS. 8A-8C, the anchoring elements 128 are circumferentially aligned relative to the bone treatment shaft or body member 120, wherein the connectors 180 are positioned cross-sectionally in the bone treatment shaft 120, as shown in FIG. 8B. The openings 140 on the bone treatment shaft 120 may be arranged circumferentially in the bone treatment shaft wall 125 and may also have a narrow configuration. The anchoring elements 128 are positioned in the openings 140, as shown in FIGS. 8A and 8B, until the outer member 170 is translated or rotated to substantially align the slots 175 with the openings 140. The rotating members 128a then rotate outwardly through the openings 140 and the slots 175, as shown in FIG. 8C. When the outer member 170 is translated or rotated over the anchoring elements 128, the anchoring elements 128 rotate inwardly back through the slots 175 to be received in the bone treatment shaft openings 140. Notably, the illustrated embodiment demonstrates rotational translation of the outer member 170 relative to the treatment shaft 120 to allow deployment and depression of the anchoring elements 128, but the anchoring elements 128 and the outer member 170 may also be configured to facilitate longitudinal translation of the outer member 170 relative to the treatment shaft 120. As illustrated in FIG. 8B, multiple anchoring elements 128 may be at the same longitudinal position on the bone treatment shaft 120, or the anchoring elements 128 may each be at different longitudinal positions on the bone treatment shaft 120. Further, as an inner member is not used to deploy the anchoring elements 128 in this embodiment, the bone treatment shaft 120 may not include a lumen 126 for receiving an inner member, such that the openings 140 are formed out of the shaft wall 125.

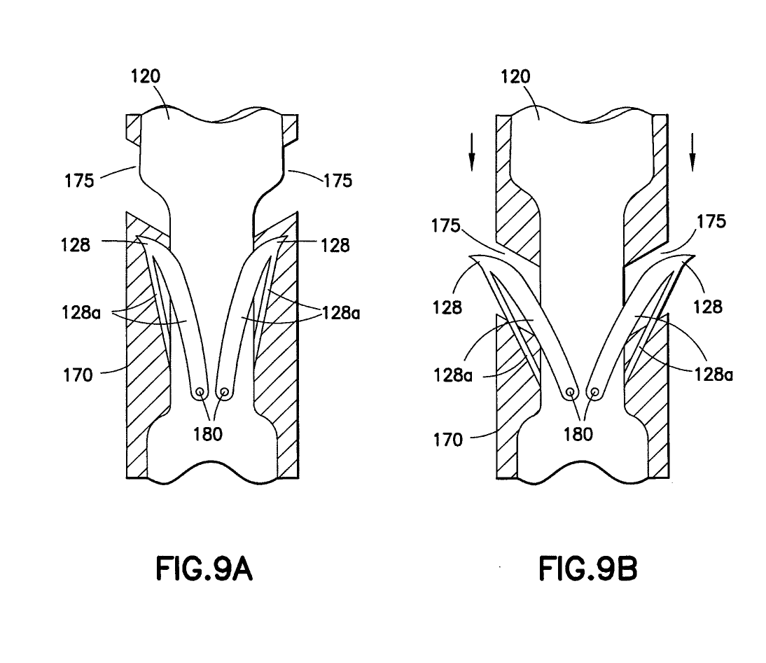

In another alternative embodiment, as shown in FIGS. 9A and 9B, each anchoring element 128 includes two joined rotating members 128a each connected to respective connectors 180. In this embodiment, at least an end portion of the rotating members 128a rotate outwardly away from the bone treatment shaft 120 and through the slots 175 when the slots 175 are aligned with the anchoring elements 128. The rotating members 128a also rotate inwardly toward the bone treatment shaft 120 when the outer member 170 is translated over the anchoring elements 128. The slots 175 may be tapered to accommodate the anchoring elements 128, as shown in FIGS. 9A and 9B. Also, the anchoring elements 128 may be arranged individually on the bone treatment shaft 120 or in pairs, as shown in FIGS. 9A and 9B.

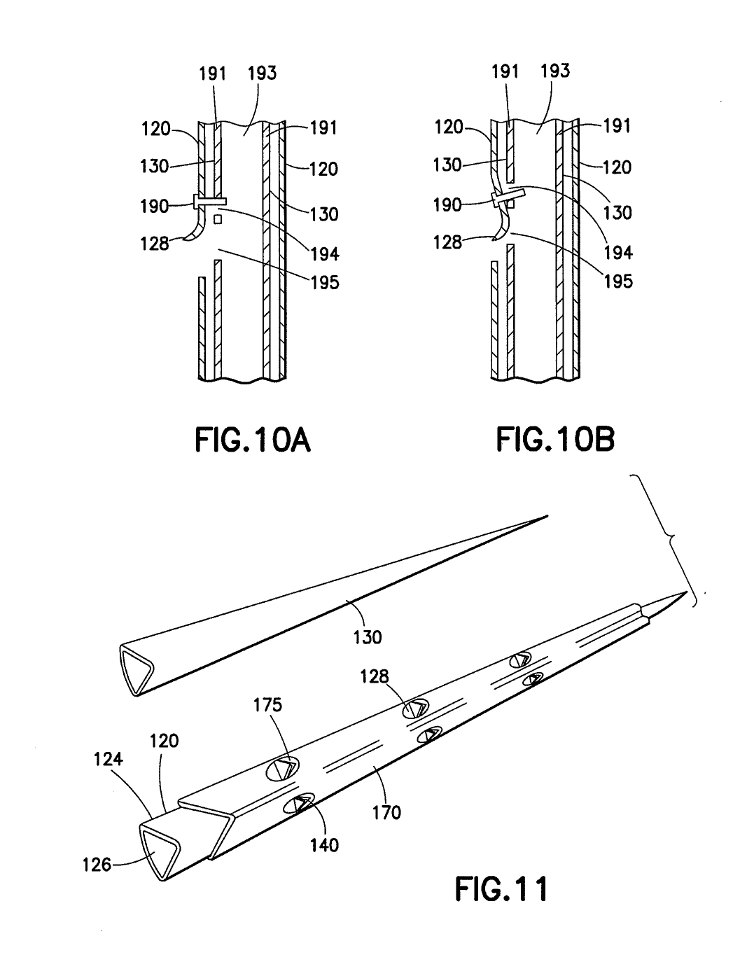

FIGS. 10A and 10B illustrate another embodiment in which the anchoring element 128 includes a sliding member 190 that extends into the bone treatment shaft lumen 126. In this embodiment, the inner member 130 has a wall 191 defining an inner lumen 193, a guiding slot 194 formed in the wall 191 for receiving the sliding member 190, and a receiving slot 195 for receiving an end portion of the anchoring element 128. When the inner member 130 is translated relative to the bone treatment shaft 120, as shown in FIG. 10A, an edge of the guiding slot 194 engages the sliding member 190, causing the sliding member 190 to translate with the inner member 130 and further causing the anchoring element 128 to deploy out of the receiving slot 195, as shown in FIG. 10A. When the inner member 130 is translated in the opposite direction relative to the bone treatment shaft or body member 120, as shown in FIG. 10B, another edge of the guiding slot 194 engages the sliding member 190, causing the sliding member 190 to translate with the inner member 130 and further causing the anchoring element 128 to retract through the receiving slot 195. The outer member 170 may not be employed in this embodiment, as the anchoring elements 128 are retained in the bone treatment shaft 120 and/or the inner member 130 by the inner member 130 contacting the sliding member 190.

While the above-described previous illustrations of the bone treatment shaft 120, inner member 130, and/or outer member 170 feature circular cross-sections, other cross-sectional shapes of the bone treatment shaft 120, inner member 130, and/or outer member 170 may be provided (e.g., triangular, rectangular, square, oval, etc.). For example, FIG. 11 illustrates a bone treatment assembly 100 which includes a treatment shaft or body member 120, inner member 130, and outer member 170 each having triangular cross-sections. Similar to the embodiment illustrated in FIG. 1, the inner member 130 is received in the bone treatment shaft lumen 126 to deploy the anchoring elements 128 through the respective openings 140 on the bone treatment shaft 120 and the slots 175 on the outer member 170. Also similar to the embodiment illustrated in FIG. 1, the outer member 170 is longitudinally translatable over the bone treatment shaft 120 to depress the anchoring elements 128 in the bone treatment shaft lumen 126. In alternative embodiments, the bone treatment shaft or body member 120, inner member 130, and outer member 170 can have other cross-sectional shapes, including elliptical, semi-circular, rectangular, square, or other customized shapes.

With a non-circular cross-section, the bone treatment shaft 120, once implanted into a bone, generally may not be rotated torsionally, i.e., about the longitudinal axis 160, such that the bone treatment shaft 120 is anchored torsionally to the bone. In such case, the anchoring elements 128 serve the purposes of anchoring the bone treatment shaft 120 to the bone, such that the bone treatment shaft 120 cannot move longitudinally within the bone, and enhancing torsional anchorage of the bone treatment shaft 120 to the bone.

In exemplary embodiments, the outer member 170 may be retained on the bone treatment shaft or body member 120. For example, one or both of the first and second ends 122, 124 of the bone treatment shaft 120 may include a stop or the like, such as a lip (not shown), to limit translation of the outer member 170. As another example, the outer member 170 may be frictionally retained on the bone treatment shaft 120 by frictional elements that prevent the outer member 170 from freely translating over the bone treatment shaft 120 without supplemental force.

In an alternative embodiment, the outer member 170 is separate from the bone treatment shaft 120, instead of being retained thereon. In this embodiment, the bone treatment shaft 120 may be inserted in a medullary canal, and the anchoring elements 128 deployed therein through the shaft openings 140 (e.g., via the inner member 130), without the outer member 170 present on the shaft 120. The outer member 170 may then be selectively translatable over the bone treatment shaft 120 from one of the first or second ends 122, 124 toward the opposite end to depress the anchoring elements 128 in the bone treatment shaft lumen 126. The bone treatment shaft 120 may then be removed if desired. Additionally, the wall 171 of the outer member 170 may be substantially continuous, i.e., without the slots 175, since the anchoring elements 128 can be deployed in the absence of the outer member 170. Thus, the outer member 170 in this embodiment may have the same dimensions and translational capability as the embodiments illustrated in FIGS. 1-4, except the slots/apertures 175 need not be included.

In exemplary embodiments, the bone treatment shaft wall 125 and/or outer member 170 may be at least partly composed of a mesh or porous material or the like with a plurality of openings (not shown) that allow for increased communication of bone tissue with the bone treatment shaft 120 and/or outer member 170 (e.g., a substantially porous interconnection structure, similar to the embodiments shown and described in U.S. Pat. Nos. 6,261,289 and 6,554,833, the entire contents of both being hereby incorporated by reference in their entireties). Bone tissue may thus grow through the plurality of openings, and bodily fluids may also flow through the plurality of openings, for increased integration of the bone treatment shaft 120 and/or outer member 170 with the surrounding bone tissue, for example. The mesh or porous material also allows for insertion of additional anchoring devices (e.g., wires or screws or the like) in the openings for additional fixation of bone fragments to the bone treatment shaft or body member 120. In the embodiment in which the outer member 170 is retained on the bone treatment shaft 120, the outer member 170 may be at least partly composed of the mesh or porous material or the like with a plurality of openings in addition to, or as an alternative to, the bone treatment shaft 120 having the mesh or porous material. The mesh or porous material of outer member 170 also allows for insertion of additional anchoring devices (e.g., wires or screws or the like) in the openings for additional fixation of bone fragments to the outer member 170. In exemplary embodiments, selected portions or the entirety of the bone treatment shaft 120 and/or the outer member 170 may also include an antibiotic or other pharmaceutical agent coating or the like to promote bone tissue health, for example.

In exemplary embodiments of the present disclosure, the bone treatment shaft 120, inner member 130, and/or outer member 170 of the assembly 100 each may be in sections or modular components (not shown) that may be coupled together, for example, by a bayonet coupling, friction fit, adhesives, or other suitable coupling structures or the like. The various sections or modular components of the assembly 100 allows for various length and diameter options of the assembly 100, with less inventory required. For example, the bone treatment shaft 120, inner member 130, and/or outer member 170 may have one end section corresponding to one of the first and second ends 122, 124 which may be provided/selected in various sizes/lengths/diameters. The remaining sections of the bone treatment shaft 120, inner member 130, and/or outer member 170 may then be provided/selected in various sizes, lengths, diameters and/or sections, so that such particularly sized sections may be selected for coupling to the selected end section(s).

Alternatively, the bone treatment shaft or body member 120, inner member 130, and/or outer member 170 may include two end sections corresponding to each of the first and second ends (e.g., ends 122, 124) with each end section provided in one size. One or more middle sections may also be provided with multiple sizes, wherein one or more of the particularly sized middle sections may be selected for coupling between the end sections. The bone treatment shaft 120 sections may also be provided with varying numbers and configurations of bone anchoring elements 128, such that a desired number and/or configuration of anchoring elements 128 may be selected. For example, this sectional embodiment provides versatility in selecting the size and structure of the assembly 100 and may also help to reduce inventory, as it may be supplied to suit different procedures and/or bone sizes, instead of supplying one bone treatment shaft for just one type of procedure or bone structure.

In another embodiment, the bone treatment assembly 100 may take the form of a telescoping bone treatment assembly 100. For example, the telescoping bone treatment assembly 100 may assist in bone lengthening procedures or the like. In one embodiment, the telescoping bone treatment assembly is actuated via a direct or telemetric actuator system. For example, the bone treatment assembly 100 may have the ability to be implanted and/or inserted along and/or inside the medullary canal of a bone at a given or certain length, and then the bone treatment assembly 100 may be incrementally lengthened to adjust the length of the assembly 100 (e.g., the length of the bone treatment shaft 120, inner member 130, and/or outer member 170) to the length of a bone segment or the like. As such, the assembly 100 may also be utilized to address a deformity and/or a segmental defect.

In exemplary embodiments, the inner member 130 may take the form of a single elongated member or actuator that is capable of deploying/displacing at least one of the anchoring elements 128. In alternative embodiments, a series of inner members 130 may be utilized to deploy/displace the anchoring elements 128. For example, each inner member 130 may take the form of a pellet sized section (not shown) which may be separate from the other sections, or the sections may be linked together, to fit within the lumen 126 of the bone treatment shaft 120 and have surfaces configured for engaging an anchoring element 128 of the bone treatment shaft 120. A user may selectively rotate each pellet as the inner member is advanced in the bone treatment shaft lumen 126 to engage and deploy a corresponding anchoring element 128. In exemplary embodiments, the single inner member 130 (or the series of inner members 130) may be configured and dimensioned so that at least one portion of the inner member 130 is configured to engage an anchoring element 128, and at least one portion of the inner member is configured to have a recess or the like that can be advanced past an anchoring element 128 without deploying that particular anchoring element 128. In other words, each inner member 130 may have one or more recesses or the like configured and dimensioned to allow a user to selectively deploy certain anchoring elements 128. For example, the inner element(s) 130 may be oriented to align each recess of the member 130 with an anchoring element 128 such that the inner element 130 may be advanced past the anchoring element 128 without deploying the anchoring element 128. Alternatively, the inner element(s) 130 may be oriented to align each engaging portion of the member 130 with an anchoring element 128 such that the inner element 130 may engage and deploy/displace the anchoring element 128 (e.g., when the member 130 is advanced in the lumen 126). Assemblies 100 having inner members 130 with such recesses and/or engaging portions have been described in U.S. patent application Ser. No. 11/036,304, the entire contents of which is expressly incorporated by reference herein.

In general, when a series of inner members 130 is utilized to deploy/displace the anchoring elements 128, each member 130 may have different configurations (e.g. different cross-sectional dimensions, different number of engaging surfaces or recesses, different lengths, etc.). Thus, different anchoring elements 128 may be deployed in different manners along the bone treatment shaft 120. In exemplary embodiments, a plunger or the like may be used to advance the inner member(s) 130 in the lumen 126. For example, as the inner member(s) are advanced in the lumen 126, the engaging surfaces of the member(s) 130 engage any anchoring elements that they come into contact with, thereby deploying/displacing the engaged anchoring elements 128 at least partially out of the lumen 126 (e.g., at least partially out of the openings 140). In exemplary embodiments, each engaged anchoring element 128 is maintained in the deployed/displaced position at least partially out of the opening(s) 140 while each engaging surface of each member 130 remains engaged with each engaged anchoring element 128, and each engaged anchoring element 128 is prevented from moving back to the pre-deployed position within the lumen 126 while each engaging surface of each member 130 remains engaged with each engaged anchoring element 128. For example, when a series of inner members 130 is utilized to deploy/displace the anchoring elements 128, each inner member 130 may be hollow and may be inserted or positioned over a guide wire and/or a cable or the like, which then may be used to pull or move the inner members 130 (e.g., for re-positioning or removal of the inner members 130).

In another embodiment, the inner member 130 includes an expanding mechanism as a means to deploy/displace the anchoring elements 128. In one embodiment, the expanding mechanism takes the form of a multi-staged cam device, although the present disclosure is not limited thereto. The multi-staged cam device may actuate the anchoring elements 128 via axial translation and/or rotation of the multi-staged cam device, as discussed below.

In another embodiment, the expanding mechanism is an expandable rolled-up tube or the like and an expander (e.g., a hydraulic or pneumatic pressure vessel, shape memory materials, etc.). For example, the expander may be positioned in the expandable tube and expanded, which causes the tube to expand and engage the anchoring elements 128, thus deploying the anchoring elements 128. Other expandable mechanisms/structures for the inner member include, without limitation, an inflatable member, a hydraulic or pneumatic pressure vessel, shape memory materials, cams, an expandable mesh, or other mechanical/electrical devices which may be inserted into the lumen 126 of the bone treatment shaft 120 and which may be expanded to deploy the anchoring elements 128. The expanding mechanism may also actuate/deploy the anchoring elements 128 by way of electronically activated devices (e.g., motors, etc.), direct energy (e.g., heat, cold), and/or by telemetric power.

In exemplary embodiments, the expanding mechanism may be an integral part of the assembly 100, or it may be removable after expansion and/or deployment of the anchoring elements 128. In one embodiment, the assembly 100 includes a locking element/mechanism which is configured and dimensioned to lock the anchoring elements 128 in the deployed position after expansion and/or deployment of the anchoring elements 128.

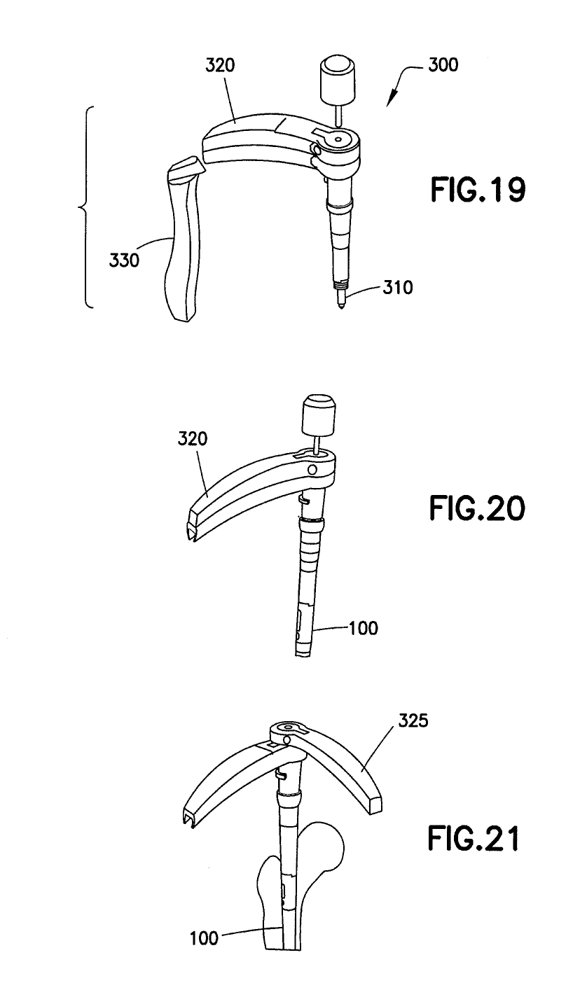

In exemplary embodiments of the present disclosure and as shown in FIGS. 19-23, at least one end section (e.g., distal and/or proximal end) of the bone treatment shaft or body member 120, inner member 130, and/or outer member 170 of the assembly 100 may be configured and dimensioned to mate and/or engage with an inserter member 300 (e.g., an inserter tool, retrieval tool, deployment member/tool, and/or an expanding mechanism/tool 300). For example and as depicted in FIGS. 19-23, at least one end section of the bone treatment shaft 120, inner member 130, and/or outer member 170 of the assembly 100 may include an engagement point or the like which is configured and dimensioned to mate and/or engage with inserter member 300.

In exemplary embodiments and as shown in FIGS. 19-23, the inserter member 300 includes at least one fixation element 310 which is configured to allow the inserter member to be releasably affixed, secured and/or coupled to the device 100. For example, the inserter member 300 may be releasably coupled to the device 100 via threads, quarter turn locks, expanding pins, etc., although the present disclosure is not limited thereto. In exemplary embodiments, the at least one fixation element 310 may be configured and dimensioned to allow for specific orientations between the inserter member 300 and device 100 (e.g., once the inserter member 300 is releasably secured to the device 100). The inserter member 300 may be disengaged from the device 100 after the device 100 is placed/positioned/deployed as desired (e.g., after final placement and/or deployment of the device in the medullary canal).

In exemplary embodiments, the inserter member 300 includes a drive mechanism or the like (e.g., a drive rod) that is configured to interface (e.g., via threads, quarter turn locks, expanding pins, etc.) with the expanding and/or deployment mechanism of the device 100. For example, the inserter member 300 may be configured to interface with the inner member 130 of the device 100 to deploy and/or un-deploy the anchoring elements 128, and/or the inserter member 300 may be configured to interface with the outer member 170 to deploy and/or un-deploy the anchoring elements 128, and/or the inserter member 300 may be configured to interface/communicate with the expanding mechanism and/or expander of the inner member 130 to deploy and/or un-deploy the anchoring elements 128. In exemplary embodiments, the drive mechanism of the inserter member 300 may be actuated in one direction (e.g., via axial translation and/or rotational movements) in order to actuate the expanding and/or deployment mechanism of the device 100. For example, the drive mechanism of the inserter member 300 may be actuated by deploying/actuating at least a first portion 325 of the handle 320 of the inserter member 300 in one direction, as depicted in FIG. 21. Such deployment/actuation of the first portion 325 of the handle 320 creates actuation of the expanding and/or deployment mechanism of the device 100 (e.g., such actuation causes the inner member 130 of the device 100 to deploy and/or un-deploy the anchoring elements 128). Conversely, the drive mechanism may be reversed in direction by reversing the direction of the first portion 325 of the handle 320 (e.g., the first portion 325 of the handle 320 may be re-positioned as depicted in FIG. 20) to deploy and/or un-deploy the anchoring elements 128. By way of example, the movement of the handle 320 (or of the first portion 325 of the handle 320) may be rotational movement about the central or longitudinal axis of the device 100, or the movement may be a cantilever movement about a pivot point at the drive end of the inserter member 300.

In one embodiment, the handle 320 may initially be in a locked form (see FIG. 20) which allows the handle to have an ergonomic shape. However, the handle may be unlocked to allow at least one section (e.g., first portion 325) of the handle 320 to actuate the drive mechanism of the inserter member 300.

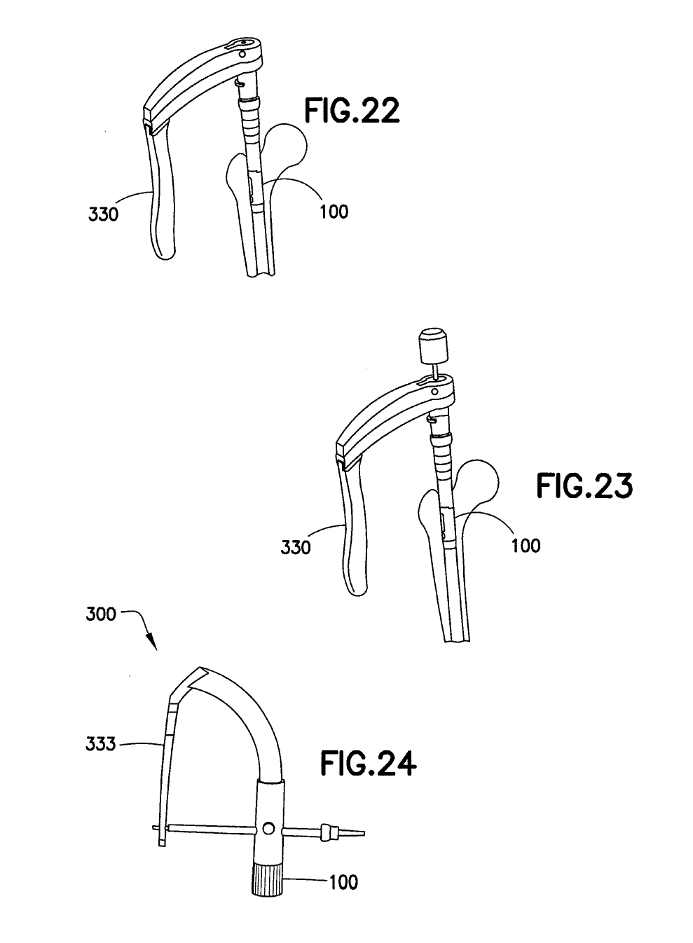

In one embodiment and as depicted in FIGS. 22-23, the inserter member 300 includes a ratchet 330 or the like. Ratchet 330 is configured to allow a user the ability to make controlled incremental movements in one direction or the other to actuate the drive mechanism of the inserter member 300.

In another embodiment and as depicted in FIG. 24, the inserter member 300 may include an attachable guide or sleeve 333 or the like which assists with the placement of screws or the like (e.g., anchoring elements) in at least one end (e.g., the proximal end) of the device 100.

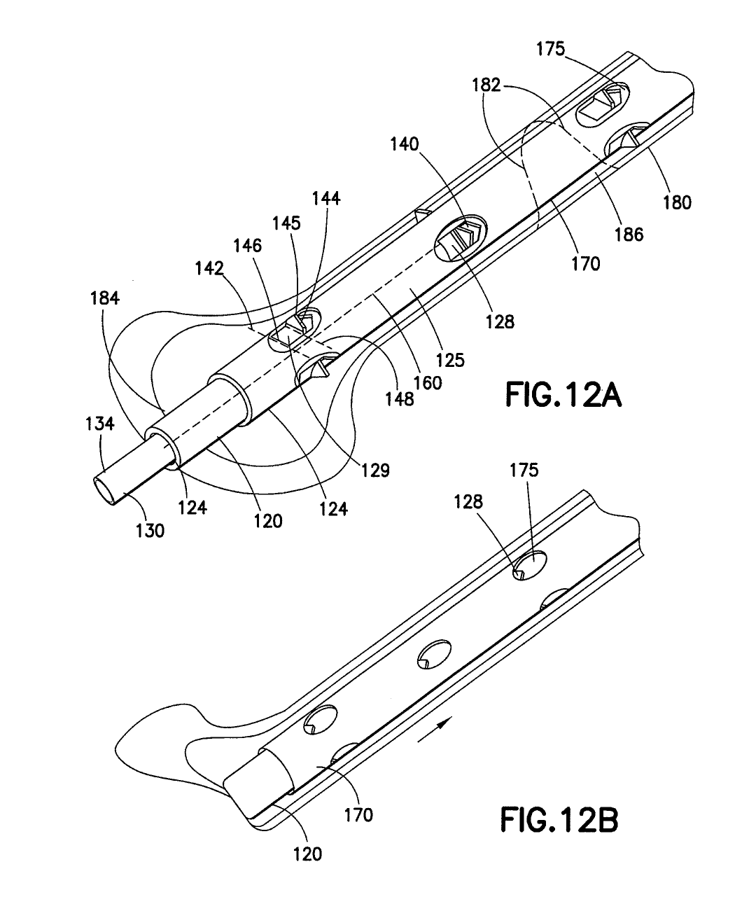

FIGS. 12A and 12B illustrate using the bone treatment assembly 100 to stabilize a femur 180 or the like having a fracture 182. For purposes of illustration, use of the bone treatment assembly 100 in FIG. 1 is described. The bone treatment shaft 120 and the outer member 170 can be inserted through a previously formed entry portal 184 into a medullary canal 186 of the femur 180 using conventional methods. Once the bone treatment shaft 120 and the outer member 170 are desirably placed, the inner member 130 can then be inserted into the lumen 126 of the bone treatment shaft 120 at the second end 124, and advanced distally to deploy the anchoring elements 128 out of the lumen 126 through the respective openings 140 of the bone treatment shaft 120 and the slots 175 of the outer member 170 (FIG. 12A). After the anchoring elements 128 are deployed, the inner member 130 may be removed, or the inner member 130 may remain in the lumen 126 to help prevent the anchoring elements 128 from being depressed back into the lumen 126.

The anchoring elements 128 penetrate bone tissue surrounding the bone treatment shaft 120 and/or the outer member 170, anchoring the bone treatment shaft 120 to the femur 180 and helping to prevent the bone treatment shaft 120 from sliding longitudinally and/or rotating about the longitudinal axis 160 relative to the femur 180. If it is later desired to remove the bone treatment shaft 120 from the femur, the outer member 170 may be translated on the bone treatment shaft 120 to dislodge the anchoring elements 128 from the bone tissue and further depress the anchoring elements 128 in the bone treatment shaft lumen 126 (FIG. 12B). If the inner member 130 is still present in the lumen 126 prior to translating the outer member 170, the inner member 130 may first be removed or re-positioned so the anchoring elements 128 are more easily depressed in the lumen 126. The bone treatment shaft 120 and the outer member 170 may then be removed from the femur 180, without the anchoring elements 128 disrupting the removal process.

Alternatively, using the embodiment in which the outer member 170 is separate from the bone treatment shaft 120, the bone treatment shaft 120 may be inserted into a medullary canal without the outer member 170. The inner member 130 may then be inserted into the lumen 126 and advanced to deploy/displace the anchoring elements 128 at least partially out of the lumen 126 and/or openings 140. When it is desired to remove the bone treatment shaft 120 from the medullary canal, the outer member 170 may then be translated over the bone treatment shaft 120 to dislodge the deployed anchoring elements 128 from the bone tissue and further depress the anchoring elements 128 in the bone treatment shaft lumen 126. Thus, this allows the outer member 170 and the bone treatment shaft or body member 120 to be removed from the medullary canal.

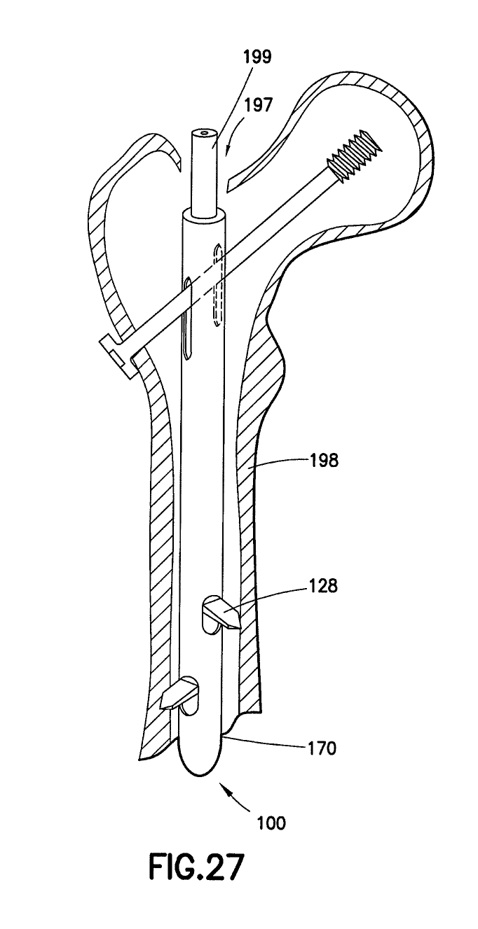

In another embodiment of the present disclosure and as shown in FIG. 27, the assembly 100 may further include an extension member 199 releasably coupled or attached to the assembly 100. In general, the extension member 199 has a smaller diameter (e.g., outer diameter) than the diameter (e.g., outer diameter) of the outer member 170 of device 100, and the extension member may be releasably coupled or attached (e.g., threadably engaged or coupled) to the proximal end of the outer member 170. Alternatively, the extension member 199 may be releasably coupled or attached (e.g., threadably engaged or coupled) to the shaft 120 and/or to the inner member 130, or to any other suitable location of device 100. In one embodiment and as illustrated in FIG. 27, the extension member 199 is releasably coupled to the proximal end of the device 100 after the device 100 has been implanted in a bone 198 (e.g., and after deployment of the anchoring elements 128), so that at least a portion of the extension member 199 extends through the bone and/or extends at least a portion through the entry portal hole 197 of the bone 198. As shown in FIG. 27, the device 100 excluding the extension member 199 is substantially buried inside the bone 198. As such, the placement of the extension member 199 at least partially extending through the bone 198 (and with the member 199 being smaller in diameter than the entry portal hole in the bone) avoids the effect of a mal-position hole that may constrain and push the device 100 to a non-desired position in the bone 198, which is a complication in some hip or proximal femur fractures. For example, after the device 100 is inserted so that it is substantially buried inside the bone 198 and while the device 100 is still attached to the inserter tool or the like (e.g., inserter or delivery system), once the desired position of the device is achieved inside the bone, then the inserter tool or the like may be removed and the extension member 199 may be releasably secured or attached (e.g., threaded) onto the proximal tip of the device, thereby closing the proximal tip of the device 100 and making it easier to locate the device 100 when removal is desired since the extension member at least partially extends out of the bone 198.

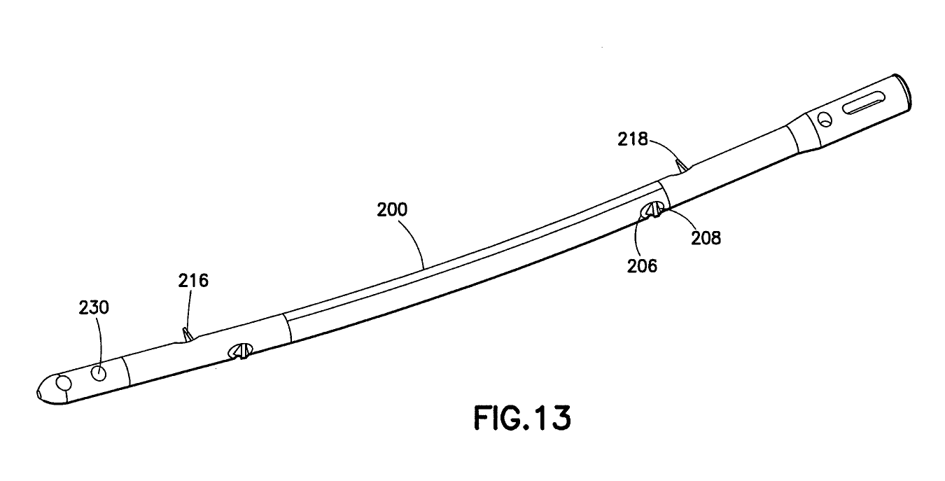

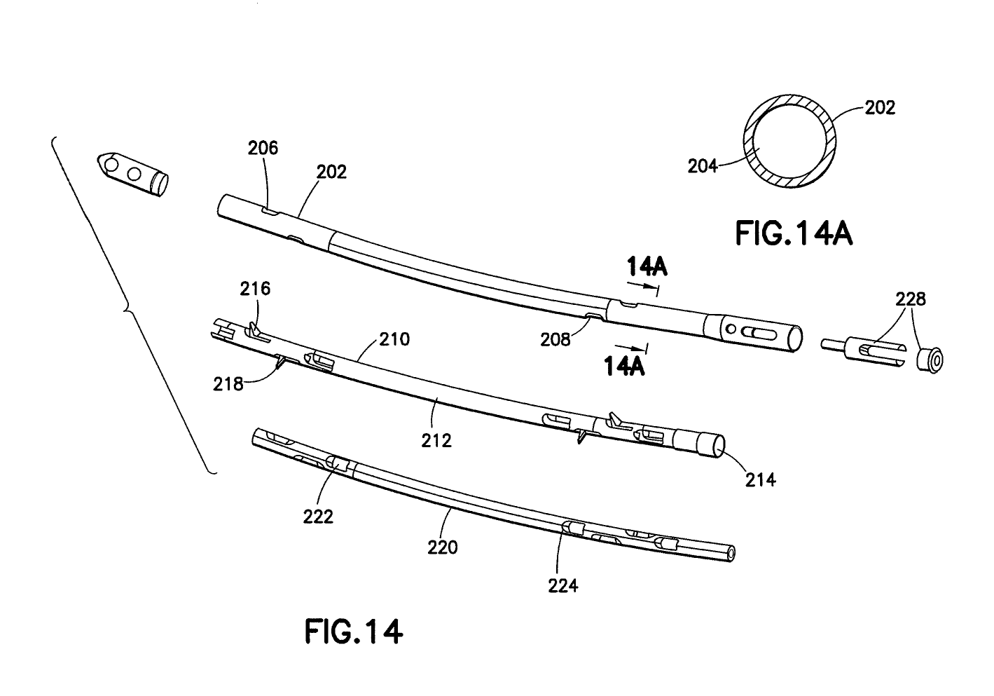

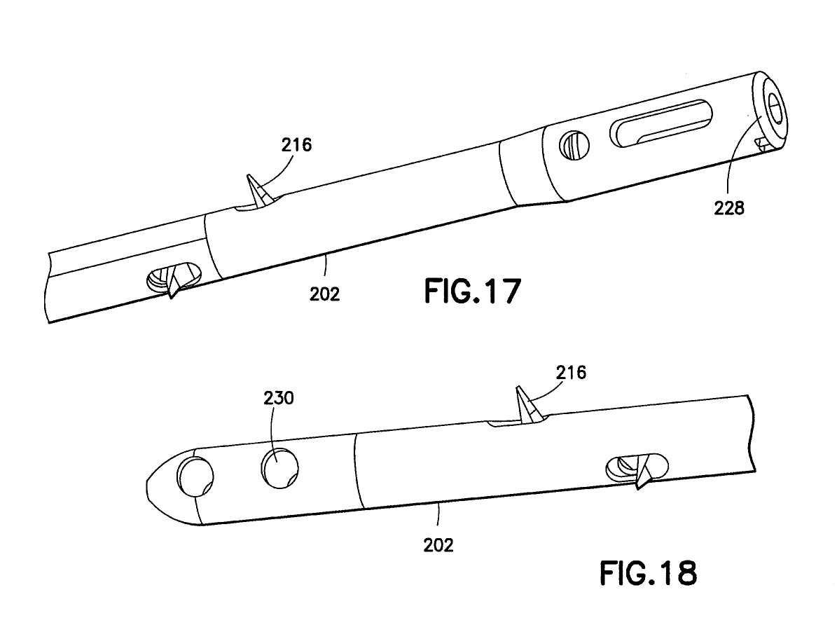

In an alternative embodiment and as depicted in FIGS. 13-18, a bone treatment device 200 (e.g., a three-layer fixation device) includes an outer shaft or member 202 (e.g., an elongated biocompatible outer shaft 202) that defines a first interior lumen 204 (see FIG. 14A). In general, the outer shaft 202 is configured and dimensioned to extend at least partially into bone tissue. In exemplary embodiments, the outer shaft 202 includes a plurality of outer openings 206 formed therein in communication with the first interior lumen 204, with each outer opening 206 being defined by a peripheral wall 208. In exemplary embodiments, the outer shaft 202 may take the form of a cannula or the like, although the present disclosure is not limited thereto. Rather, the outer member 202 may take a variety of forms.

Typically, the second or middle layer of the device 200 is an anchoring shaft or body member 210 which is configured and dimensioned to be received and/or positioned within the first interior lumen 204 of the outer shaft 202. In exemplary embodiments, the anchoring shaft 210 includes a shaft wall 212 defining a second interior lumen 214 and also includes bone anchoring elements 216 formed thereon or coupled/connected to the anchoring shaft wall 212.

In exemplary embodiments, the shaft or body member 210 includes a plurality of bone anchoring elements 216 coupled (e.g., hingedly coupled) and/or connected to the shaft wall 212 and a plurality of respective openings formed through the shaft wall 212. In one embodiment, each anchoring element 216 has a first end having a tissue-piercing portion (e.g., a sharp, tissue-piercing tip or the like), and a second end that is coupled, secured and/or connected to the wall 212 of the shaft or body member 210. For example, the bone anchoring elements may be similar to the bone anchoring elements 128 discussed above in relation to FIGS. 1-3.

In general, the bone anchoring elements 216 are deployable/displaceable through the outer openings 206 of the outer shaft 202 to penetrate/engage bone and/or bone tissue adjacent to the outer shaft 202. The bone anchoring elements 216 may also be retracted in the second interior lumen 214 of the anchoring shaft 210.

As described above for the other embodiments, the bone anchoring elements 216 may be formed out of the anchoring shaft wall 212, or secured, coupled and/or connected to the anchoring shaft 210 by other means (e.g., hingedly coupled or connected, via a coupling/connection device and/or adhesive or the like, welded, etc). In one embodiment, the bone anchoring elements 216 are manufactured from the same/similar material as the anchoring shaft 210. To facilitate bone penetration, the bone anchoring elements 216 may be bent at any suitable angle and may have a tissue-piercing portion 218 (e.g., tissue-piercing tips or the like), as shown in FIGS. 13 and 14.

In exemplary embodiments, the third or inner layer of the device 200 is an actuator or inner member 220 (e.g., a drive rod or the like) which is configured and dimensioned to be received and/or positioned within the second interior lumen 214 of the anchoring shaft 210. In general, the inner member 220 includes a plurality of inner openings or recesses 222 (e.g., excavated portions 222), with each inner opening or recess 222 being defined by a peripheral wall 224. In one embodiment, the inner member 220 is hollow, so that the inner member 220 of the device 200 is capable of being implanted and/or positioned over a guide wire or the like. In exemplary embodiments, each inner opening/recess 222 is configured and dimensioned to receive or house part or substantially all of at least one of the bone anchoring elements 216 within the inner opening/recess 222. For example, the inner member 220 may be inserted/positioned into the second interior lumen 214 of the anchoring shaft 210 so that each bone anchoring element 216 substantially aligns with an opening/recess 222 of the inner member 220. In an exemplary embodiment, each bone anchoring element 216 may then be placed or positioned (e.g., manually pushed or forced or the like) in an opening or recess 222 of the inner member 220. At this point, the bone anchoring elements 216 are undeployed (e.g., in the undeployed position), and the anchoring shaft 210 (with the inner member 220 inserted/positioned therein) may take the form of a generally cylindrical tube or the like with substantially no bone anchoring elements 216 protruding from the anchoring shaft 210. The anchoring shaft 210 and inner member 220 sub-assembly may then be inserted/positioned within the first interior lumen 204 of the outer shaft 202 so that each bone anchoring element 216 substantially aligns with an outer opening 206 of the outer shaft 202.

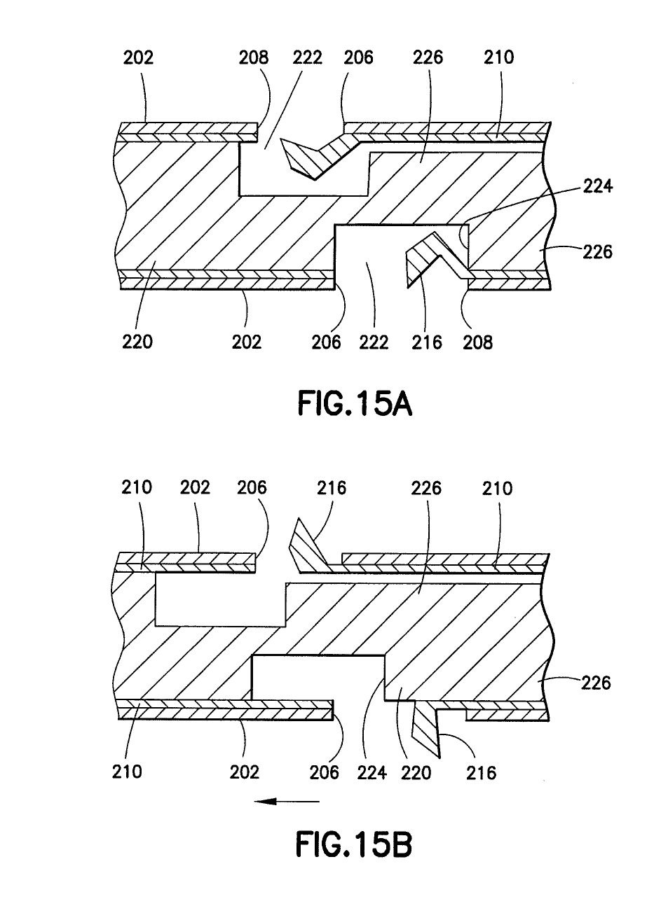

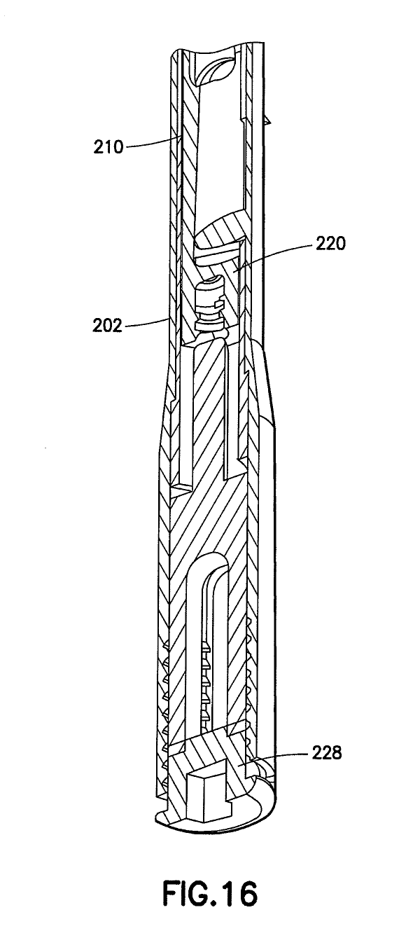

In one embodiment and as depicted in FIGS. 15A and 15B, the inner member 220 may be translated (e.g., axially) to outwardly displace the bone anchoring elements 216 through the outer openings 206 in the outer shaft 202 to penetrate/engage bone and/or bone tissue adjacent to the outer shaft 202. For example, in the illustrated embodiment, as the inner member 220 is distally translated, the walls 224 of the inner openings/recesses 222 contact the bone anchoring elements 216 and urge the bone anchoring elements 216 at least partially through the outer openings 206, e.g., similar to a cam mechanism, causing the bone anchoring elements 216 to penetrate adjacent bone tissue. In one embodiment, following displacement of the bone anchoring elements 216 at least partially through the outer openings 206, a continuous surface 226 of the inner member 220 adjacent to each of the inner openings 222 is then positioned under the bone anchoring elements 216 to support the outward displacement of the bone anchoring elements 216. This also helps stabilize the position of the device 200, as the continuous surface 226 helps maintain the position of the bone anchoring elements 216 in bone tissue and prevents the bone anchoring elements 216 from retracting into the inner openings 222. Notably and in one embodiment, the outer shaft 202 does not substantially move in relation to the anchoring shaft 210 during deployment of the bone anchoring elements 216 (e.g., the outer shaft 202 and the anchoring shaft 210 are preferably passive and do not translate or displace relative to one another as the inner member 220 is distally translated to deploy the bone anchoring elements 216). In an exemplary embodiment, once deployed, the bone anchoring elements 216 are oriented at a suitable angle (e.g., about 90°) relative to the longitudinal axis of the device 200. It is to be noted that the bone anchoring elements 216 may be oriented at any suitable angle relative to the longitudinal axis of the device 200 once deployed.

In one embodiment, with an inverse movement of the inner member 220, the bone anchoring elements 216 are able to retract inwardly back through the respective outer openings 206 in the outer layer. For example, in one embodiment, the inner member 220 is proximally translated such that the inner openings 222 are adjacent to the bone anchoring elements 216, similar to the position illustrated in FIG. 15A, allowing the bone anchoring elements 216 to be received therein. In an exemplary embodiment, once the inner openings 222 are substantially realigned with the bone anchoring elements 216, the outer shaft 202 may be translated distally to force/urge (e.g., via the peripheral walls of the openings 206) the bone anchoring elements 216 into the aligned inner openings 222 of the inner member 220. Once the anchoring elements are returned to their undeployed positions, the device 200 may be removed from and/or re-positioned in the medullary canal.

In another embodiment, the anchoring shaft 210 may also be proximally translated such that the bone anchoring elements 216 are caused to be displaced from the bone tissue into the inner openings 222. For example, after deployment of the anchoring elements 216 as discussed above, the inner openings 222 may then be substantially realigned with the anchoring elements 216. The anchoring shaft 210 may then be translated proximally, causing the bone anchoring elements 216 to contact the walls of the outer openings 206 (while the outer shaft 202 remains stationary), and the bone anchoring elements 216 are thereby displaced into the inner openings 222 due to the resistive force of the outer opening walls 208. When the bone anchoring elements 216 are retracted in the inner openings 222, the device 200 may be removed from the bone.

In an alternative embodiment, proximal translation of the inner member 220 causes the bone anchoring elements 216 to at least be partially displaced through the outer openings 206, and distal translation of the outer member 202 causes the bone anchoring elements 216 to be retracted into the inner openings 222. In yet another alternative embodiment, the device 200 is configured and dimensioned such that rotating the inner member 220 in opposing directions causes the bone anchoring elements 216 to respectively be outwardly displaced and then retracted through the outer openings 206.

In one embodiment, the device 200 includes a displacement cap or member 228 (e.g., a drive cap 228), as illustrated in FIGS. 14, 16, and 17. The displacement cap 228 may be configured and dimensioned to be insertable in the first interior lumen 204 of the outer shaft 202, and/or insertable in the second interior lumen 214 of the anchoring shaft 210, as shown in FIG. 16. In one embodiment, the displacement cap 228 is inserted in the first interior lumen 204 of the outer shaft 202 until the displacement cap 228 engages the inner member 220 and translates the inner member 220 axially (e.g., distally). For example, when the displacement cap 228 is inserted/translated, the displacement cap 228 contacts/engages the inner member 220 and causes the inner member 220 to longitudinally translate and displace the bone anchoring elements 216 through the outer openings 206, as described above. In an exemplary embodiment and as depicted in FIG. 16, a distal end of the displacement cap 228 is coupled/engaged to the proximal end of the inner member 220 by mating threads or the like, or by any other suitable type of coupling mechanism/method. In another embodiment, the distal end of the displacement cap 228 abuts the proximal end of the inner member 220. Also, a portion of the displacement cap 228 may have threads mating with threads in the first interior lumen 204 of the outer shaft 202, as shown in FIG. 16, such that the displacement cap 228 translates while being rotated along the threads of the outer shaft 202. In another embodiment, a portion of the displacement cap 228 may have threads mating with threads in the second interior lumen 214 of the anchoring shaft 210 such that the displacement cap 228 translates while being rotated along the threads of the anchoring shaft 210.

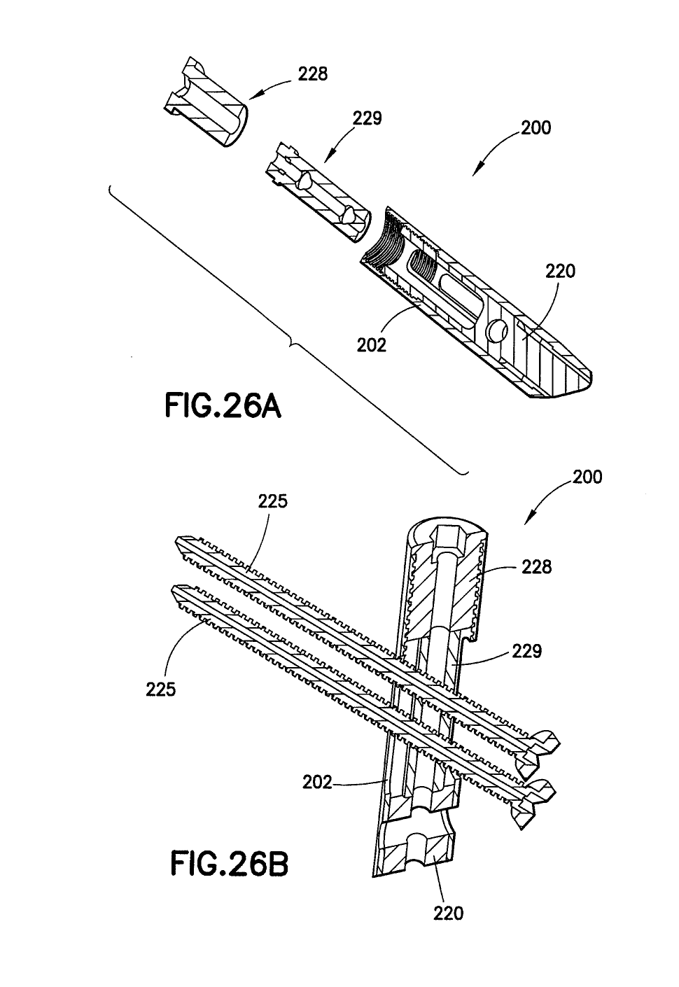

In one embodiment and as shown in FIGS. 26A-26B, the assembly 200 may further include a screw guide insert 229 or the like. In exemplary embodiments, the screw guide insert 229 is positioned between the displacement cap 228 and the inner member 220, although the present disclosure is not limited thereto. For example, the displacement cap 228 may be inserted in the first interior lumen 204 of the outer shaft 202 until the displacement cap 228 and/or the screw guide insert 229 engages the inner member 220 and translates the inner member 220 axially to displace or deploy the bone anchoring elements 216. In general and as shown in FIG. 26B, the outer shaft 202, the inner member 220, and/or the screw guide insert 229 may be configured and dimensioned to then allow at least one screw 225 or the like to be inserted through the screw guide insert 229, to thereby further help stabilize the device 200. In other words, once the assembly 200 is inserted or implanted in a bone and after the displacement cap 228 is inserted into the assembly 200 (e.g., to deploy the bone anchoring elements, as discussed above), a user may insert at least one screw 225 or the like through the screw guide insert 229 to further stabilize the device 200.

In general, a user of the assembly 200 may choose the appropriate screw guide insert 229 to be utilized in conjunction with the size, length and/or diameter of each assembly 200. In other words, a user (e.g., a surgeon) may first decide what size assembly 200 (e.g., length and/or diameter) to use, and then the user may select from multiple screw guide insert 229 options to utilize (e.g., a user may select from several different screw guide inserts 229 that may be utilized with the selected assembly 200, with each screw guide insert 229 having a different screw pattern and/or screw size option or options). For example, the screw guide insert 229 may come in many different configurations to address specific fractures and may be selected after the assembly 200 has been implanted or inserted into the bone. In this way, the multiple screw guide insert 229 options/configurations available to a user offer the user the ability to select from various screw angles, as well as how many screws can be inserted through the screw guide insert 229. Thus, this alleviates the need to offer a specific assembly size for every screw configuration (e.g., proximal screw configuration) that a surgeon may choose intra-operatively, which thereby decreases the amount of inventory a company or manufacturer would need to manufacture, as well as decreases the amount of inventory that a user (e.g., a surgeon) needs to have available when performing surgical cases, which thereby provides a significant commercial and manufacturing advantage as a result.

In one embodiment, to retract the bone anchoring elements 216, the displacement cap 228 is removed from the device 200 to allow access to the inner member 220 and the anchoring shaft 210. The proximal end of the inner member 220 may then be grasped and proximally translated to substantially align the inner openings 222 with the bone anchoring elements 216. The proximal end of the anchoring shaft 210 may then be grasped and pulled, causing the bone anchoring elements 216 to be retracted in the inner openings 222, as described above. Alternatively, once the inner openings 222 are substantially realigned with the bone anchoring elements 216, the outer shaft 202 may be translated distally to force/urge (e.g., via the peripheral walls of the openings 206) the bone anchoring elements 216 into the aligned inner openings 222 of the inner member 220. Once the anchoring elements are returned to their undeployed positions, the device 200 may be removed from and/or re-positioned in the medullary canal.

In the embodiment in which the displacement cap 228 is coupled to the inner member 220, the displacement cap 228 may be proximally translated, causing proximal translation of the inner member 220 and further causing the inner openings 222 to align or re-align with the bone anchoring elements 216. The inner member 220 and the anchoring shaft 210 may also be configured such that the inner member 220 engages the proximal end of the anchoring shaft 210 within the anchoring shaft interior lumen 214. For example, the anchoring shaft interior lumen 214 may have a narrowed cross-section that the inner member 220 contacts. Thus, as the inner member 220 is proximally retracted with the displacement cap 228, the inner member 220 engages the anchoring shaft 210, causing the anchoring shaft 210 to also proximally translate. The bone anchoring elements 216 are then retracted in the inner openings 222 upon contacting the walls 208 of the outer openings 206, as described above.

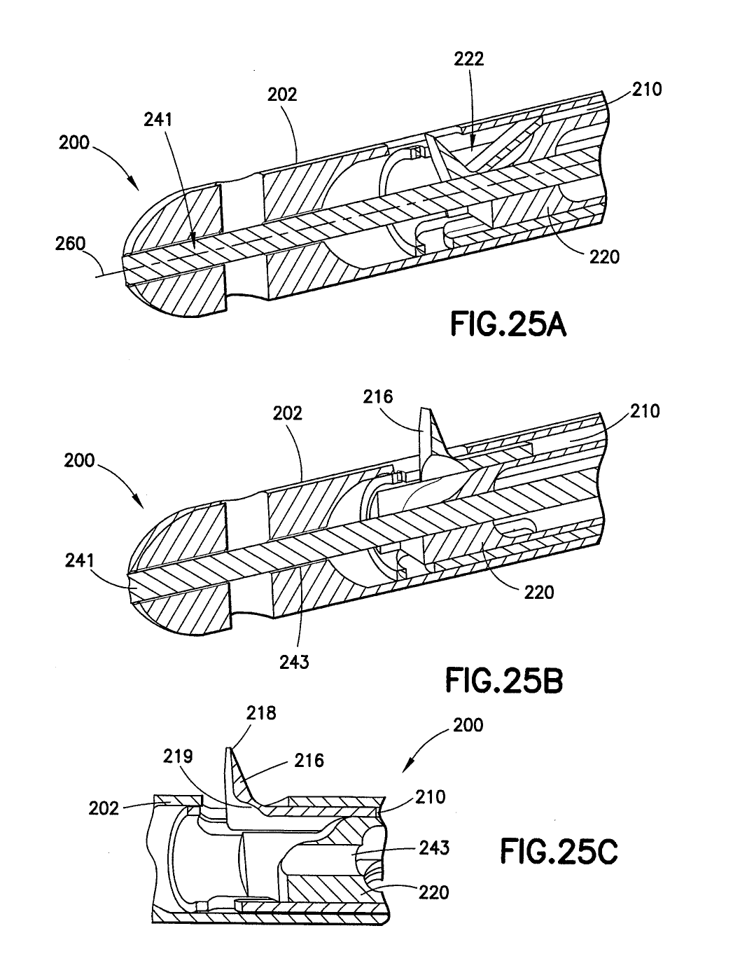

In exemplary embodiments and as depicted in FIGS. 25A-25C, the bone treatment assembly 200 is configured and dimensioned to allow a guide wire 241 or the like to be inserted and/or disposed within the assembly 200 (e.g., during the insertion of the assembly 200 into a given long bone). In general, the inner member 220, the anchoring shaft 210, the outer member 202 and/or the bone anchoring elements 216 may be configured and dimensioned to allow a guide wire 241 or the like to be inserted and/or disposed within the assembly 200. In one embodiment, the assembly 200 is configured and dimensioned to allow a guide wire 241 or the like to be inserted within and/or disposed along the central longitudinal axis 260 of the assembly 200. For example and as shown in FIGS. 25A-25C, the assembly 200 (e.g., at least the inner member 220 and the outer member 202 of the assembly 200) includes a cannulation 243 or the like (e.g., along the central longitudinal axis 260) which allows for a guide wire 241 or the like to be inserted and/or disposed within the assembly 200. In addition and as illustrated in FIGS. 25A-25C, the bottom-side 219 of each bone anchoring element 216 may be configured and dimensioned to conform to the shape of the cannulation 243 or the like when in the un-deployed position, thereby allowing the guide wire 241 or the like to be unimpeded when inserted or disposed in the assembly 200. As such and in exemplary embodiments of the present disclosure, each bone anchoring element 216 may be deployed or un-deployed with or without the guide wire 241 or the like present in the assembly 200. Additionally, this allows for variable lengths of bone anchoring elements 216 to be utilized with the assembly 200, as the bottom-side 219 of each different length bone anchoring element may be configured and dimensioned to conform to the shape of the cannulation 243 or the like when in the un-deployed position, thereby allowing the guide wire 241 or the like to be unimpeded when inserted or disposed in the assembly. In other words and as shown in FIGS. 25a-25C, bone anchoring elements 216 having a length that cause them to cross the center longitudinal axis 260 line of the assembly when in the un-deployed position may be utilized in conjunction with a guide wire 241 or the like and still not impede the passage of the guide wire 241 or the like, as the bottom-side 219 of such bone anchoring elements may be configured and dimensioned to conform to the shape of the cannulation 243 or the like. Moreover, such bone anchoring elements 216 may be deployed or un-deployed with or without the guide wire 241 present in the assembly 200.

In alternative embodiments, an inserter member 300 (see FIGS. 19-23) may be releasably secured to the device 200, and the inserter member 300 may be operated/utilized as discussed above in relation to device 100 to deploy and/or un-deploy the bone anchoring elements 216 of device 200. In addition, the assembly 200 may include a screw guide insert 229 (FIGS. 26A-26B) positioned between an inserter member 300 and the inner member 220 (e.g., one may use an inserter member 300 in place of a displacement cap 228 of assembly 200). Other features and/or functions of the inserter member 300 may be utilized in conjunction with the device 200 as well, as discussed above.

It is also to be noted that an extension member 199 (FIG. 27) may be releasably secured or attached to the device or assembly 200, and the extension member 199 may be utilized in conjunction with assembly 200, as discussed above in relation to device 100. For example, an extension member 199 (FIG. 27) may be releasably secured or attached to the outer member 202 of assembly 200. Alternatively, an extension member 199 may be releasably secured or attached to the shaft 210, the inner member 220, and/or to the displacement cap 228, or to any other suitable location on assembly 200. By using the extension member 199, the assembly 200 may be substantially buried inside a treated bone, and the extension member 199 will indicate the entry portal and the assembly 200 location (e.g., for future removal purposes, thereby avoiding possible mal-position in cases of poor entry portal location).

The device 200 may also include holes 230 extending through all layers/members of the device 200 at one or both ends of the device 200, as illustrated in FIGS. 13 and 18. The holes 230 may accommodate screws or other instruments to help stabilize the device 200 in an intramedullary canal or to provide access to tools used for inserting or removing the device 200 from bone tissue. It is to be noted that device or assembly 100 may also include holes 230, which may accommodate screws or other instruments to help stabilize the device 100 in an intramedullary canal or to provide access to tools used for inserting or removing the device 100 from bone tissue.

While use of the above-described bone treatment devices to treat a femur has been described, it should be appreciated that the disclosed and described bone treatment devices may also be used to treat other bones, such as, for example, a tibia, a humerus, a vertebra through a pedicle, etc. The bone treatment devices may thus may vary in size/shape (e.g., diameter, length), as described above, to accommodate different bone sizes and structures. For example, the bone treatment device may be flared, i.e., have a larger cross-section, at the ends to conform to the medullary canal of a femur. As another example, the bone treatment device may be narrow and tapered to conform to the medullary canal of a tibia. The bone treatment device may also be straight or curved according to the anatomy of the bone.

In addition to the above-described devices 100 and 200, the present disclosure provides for bone treatment devices (including the anchoring elements, and with or without additional holes) which may be fabricated in a short version for the treatment of fractures of the ends of the bones, such as, for example, hip fractures (all types), or distal femur fractures, distal radius, proximal or distal humerus, proximal ulna, clavicle, metatarsal or metacarpal and others. Device 100 or 200 may also be utilized for the fusion of joints (e.g., in the ankle, wrist, etc.), or longer versions for knee fusion. In addition, in appropriately sized versions, device 100 or 200 may be used to attach other devices to fractured or intact bones, such as endo-prosthesis for replacement of joints (e.g., hip, knee, shoulder, elbow, ankle), with or without the use of bone cement. For example, a joint replacement part may be attached to device 100 or 200, or it can be manufactured as an integral part of the device. In such cases, the anchoring elements will secure the attachment of the device to the bone, leaving the joint surface area of the device free to interact with the other side of the joint, or with similar artificial surfaces to form a total joint replacement device. Another exemplary application of device 100 or 200 is to attach the device to bone sutures, ligaments, tendons or combinations of such structures.

Although the systems and methods of the present disclosure have been described with reference to exemplary embodiments thereof, the present disclosure is not limited to such exemplary embodiments and/or implementations. Rather, the systems and methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the appended claims be construed broadly and in a manner consistent with the scope of the disclosure.

1. A bone treatment assembly comprising:
an outer member defining a first lumen; an elongated body member configured and dimensioned to be at least partially disposed within the first lumen, the elongated body member defining a second lumen and at least one opening through the body member, the at least one opening being in communication with the second lumen; at least one bone anchoring element coupled to the body member adjacent the at least one opening, the at least one bone anchoring element being configured and dimensioned to be moveable between at least: (i) a first position at least partially inside the second lumen through the at least one opening to allow positioning of at least a portion of the body member into bone tissue, and (ii) a second position at least partially out of the second lumen and the at least one opening to engage bone tissue adjacent thereto; an inner member configured and dimensioned to be at least partially disposed within the second lumen to engage the at least one anchoring element, thereby moving the at least one anchoring element from the first position to the second position; and wherein after the inner member has moved the at least one anchoring element from the first position to the second position, the outer member is configured and dimensioned to be translatable relative to the body member to thereby move the at least one anchoring element from the second position to the first position.

2. The assembly of claim 1, wherein the body member and the outer member have circular or non-circular cross-sections.

3. The assembly of claim 1, wherein the outer member is longitudinally or rotationally translatable relative to the body member.

4. The assembly of claim 1, wherein the outer member is selectively separable from the body member.

5. The assembly of claim 1, wherein the outer member further comprises at least one slot through the outer member, the at least one slot configured and dimensioned to be substantially aligned with the at least one opening through the body member; and wherein the at least one anchoring element is configured and dimensioned to be moved at least partially out of the at least one slot when the at least one slot is substantially aligned with the at least one opening and the at least one anchoring element is moved to the second position.

6. The assembly of claim 5, wherein the outer member includes a surface adjacent to the at least one slot, the surface configured and dimensioned to move the at least one anchoring element from the second position to the first position when the outer member is translated relative to the body member.

7. The assembly of claim 5, wherein the at least one slot includes an extension extending from the at least one slot, and wherein the outer member is configured and dimensioned to be rotationally and longitudinally translated such that the at least one anchoring element is positioned in the extension to maintain the at least one anchoring element in the second position.

8. The assembly of claim 1, wherein the at least one anchoring element includes a hinged portion extending along a first axis and an engaging portion formed by bending an end of the hinged portion along a second axis, the second axis being substantially perpendicular to the first axis.

9. The assembly of claim 1, wherein the at least one anchoring element includes a hinged portion extending along a first axis and an engaging portion formed by bending an end of the hinged portion along a second axis, the second axis being angled relative to the first axis.

10. The assembly of claim 1, wherein the at least one anchoring element is formed out of the body member.

11. The assembly of claim 1, wherein the at least one anchoring element is at least partially plastically deformable.

12. The assembly of claim 1, wherein the inner member is configured and dimensioned to expand within the second lumen to engage and move the at least one anchoring element from the first position to the second position.

13. The assembly of claim 1, wherein the inner member is configured and dimensioned to slide within the second lumen to engage and move the at least one anchoring element from the first position to the second position.

14. The assembly of claim 1, wherein the inner member is configured and dimensioned to translate within the second lumen relative to the body member in a first direction to engage and move the at least one anchoring element from the first position to the second position.

15. The assembly of claim 14, wherein the inner member is configured and dimensioned to translate within the second lumen relative to the body member in a second direction to move the at least one anchoring element from the second position to the first position.

16. The assembly of claim 1, wherein the at least one anchoring element further includes a sliding member, the sliding member configured and dimensioned to be received in a guiding slot in a wall of the inner member.

17. The assembly of claim 1, wherein the body member is at least partly comprised of a mesh.

18. The assembly of claim 1, wherein the body member or the outer member is at least partially coated with a pharmaceutical agent.

19. The assembly of claim 1, wherein the at least one anchoring element has a first end having a tissue-piercing portion and a second end that is coupled to a wall of the body member.

20. The assembly of claim 1, wherein the engaged inner member maintains the at least one anchoring element in the second position and prevents the at least one anchoring element from moving to the first position; and wherein the inner member is moved out of engagement with the at least one anchoring element prior to translating the outer member relative to the body member to move the at least one anchoring element from the second position to the first position.

21-63. (canceled)
